(19) United States
(12) Patent Application Publication
(10) Pub. No.: US 2022/0238233 A1
(43) Pub. Date: Jul. 28, 2022

(54) EPIGENETICS-BASED HEALTH AND DISEASE ASSESSMENTS FOR TREATMENT AND WELLNESS RECOMMENDATIONS

(71) Applicant: Prosper DNA Inc., Calabasas, CA (US)

(72) Inventors: Richard HAMILTON, Calabasas, CA (US); Dennis MONTOYA, Sacramento, CA (US); Matteo PELLEGRINI, Sherman Oaks, CA (US)

(21) Appl. No.: 17/611,778

(22) PCT Filed: May 21, 2020
(86) PCT No.: PCT/US20/33993

§ 371 Date: Nov. 16, 2021

Related U.S. Application Data
(60) Provisional application No. 62/851,926, filed on May 23, 2019.

(51) Int. Cl. G16H 50/30; G16H 10/60; G16H 10/40; G16H 20/00

(52) U.S. Cl. CPC G16H 50/30; G16H 20/00; G16H 10/40; G16H 10/60

(57) ABSTRACT

Systems, methods, and devices are disclosed for providing personalized wellness recommendations. Epigenetic data is obtained for a person, and wellness goals can be identified for the person. Based on the person's epigenetic data and wellness goals, one or more wellness recommendations can be determined. An indication of the wellness recommendations can then be provided to the person or a third-party.

### CROSS-REFERENCE TO RELATED APPLICATIONS

This application claims the benefit of priority to U.S. Application Ser. No. 62/851,926, filed May 23, 2019, the disclosure of which is considered part of the disclosure of the present document and is incorporated by reference in its entirety.

### BACKGROUND

Epigenetics relates to the study of gene expression in organisms apart from changes in the organism's underlying DNA sequence. While a DNA sequence is static and substantially fixed for the duration of an organism's lifetime, epigenetic patterns in contrast can change and are impacted by environmental and behavioral factors. For example, studies have shown that epigenetic patterns can be used to predict a person's age and assess whether the person's “biologic” age lags or leads the person's true, chronologic age. Similarly, studies have shown that epigenetic patterns can be used to predict a person's risk of all-cause mortality, and to estimate smoking and drinking habits of individuals due to changes that these activities impart on epigenetic patterns.

One method for analyzing epigenetic patterns involves identification of methylation sites and patterns in an individual's DNA sequence. Methyl groups (CH₃) have been observed to bond to nucleotides in the DNA sequence based on an individual's epigenetics, thereby “methylating” the DNA sequence. While methylation does not affect the underlying genetics of the individual (i.e., does not impact the sequence of nucleotides in the individual's DNA), it can nonetheless affect gene expression and the activation of certain genes in the DNA sequence. For example, heavy methylation can prevent effective transcription of genes near the methylated portion of the DNA. As environmental and behavioral characteristics change, the individual's methylation pattern can also change, thus causing changes in gene expression that correlate to environmental and behavioral factors.

### SUMMARY

Systems, methods, devices, and other techniques are disclosed for assessing the health or wellness of a person based on information about a person's epigenetics. Some implementations include a method for providing personalized wellness recommendations. The method can include obtaining epigenetic data for a person, identifying wellness goals for the person, and determining one or more wellness recommendations for the person based on the epigenetic data and the wellness goals for the person. An indication of the one or more wellness recommendations is provided to the person or another entity, such as an insurance carrier or healthcare provider of the person. Additionally, or alternatively, the method can include assessing a condition of one or more diseases for the person based on the epigenetic data, and determining one or more treatments or therapies for the person with respect to the one or more diseases, the treatments determined based on at least one of the assessed conditions of the one or more diseases, the epigenetic data, the wellness goals for the person, the genetic data for the person, biographical information for the person, or genetic data for the person.

These another other implementations can optionally include one or more of the following features.

The method can further include obtaining genetic data for the person and determining the one or more wellness recommendations for the person further based on the genetic data. The genetic data can include polygenic scores, for example.

The method can further include obtaining biographical information for the person, and determining the one or more wellness recommendations for the person further based on the biographical information. The biographical information can include information indicating at least one of an age, gender, ethnicity, height, weight, body-mass index (BMI), waist circumference, wrist circumference, or medical history of the person, for example. The biographical information can also incorporate an indication of whether a person regularly uses a fitness tracker and data obtained from the fitness tracker, an indication of whether the person (e.g., with diabetes) regularly monitors blood glucose levels and data obtained from such monitoring.

The epigenetic data can describe a methylation pattern, or other epigenetic modifications, in DNA of the person. The DNA can be extracted from a biological sample from the person, and the biological sample can be saliva, blood, or hair, for example.

The epigenetic data can describe one or more epi-alleles in DNA of the person.

The wellness goals can be selected from a group that includes goals to live longer, have more energy, be happier, sleep better, increase muscle tone, improve cardiovascular fitness, reduce back, joint, or muscle pain, improve nutrition, lose weight, reduce or quit alcoholic drinking, reduce or quit smoking, improve pre-natal health, improve pen-natal health, improve post-natal health, treat one or more particular diseases, prevent onset of one or more particular diseases, and improve sex life.

The method can further include assigning weights to the wellness goals and determining the wellness recommendations for the person using the weights for the wellness goals.

Determining wellness recommendations for the person can include determining recommendations for the person in one or more wellness categories. Wellness recommendations can additionally, or alternatively, include recommendations to consult with a physician, e.g., to follow-up on epigenetic test results that indicate an elevated risk or diagnosis of one or more diseases.

The method can further include selecting the one or more wellness categories from a plurality of wellness categories based on at least one of the wellness goals for the person, the epigenetic data for the person, biographical information for the person, or genetic data for the person.

The wellness categories can include at least one of a nutrition category, a fitness category, or a mindfulness category.

The wellness categories can include a nutrition category, and the wellness recommendations can include a recommendation for a suggested nutrient, food, or food group for the person to consume to improve nutrition.

The wellness categories can include a fitness category, and the wellness recommendations can include a recommendation for a suggested fitness activity or type of fitness activity for the person to engage in to improve fitness.

The wellness categories can include a mindfulness category, and the recommendations can include a recommendation for a suggested mindfulness activity or mindfulness routine for the person to engage in to improve mindfulness.

The method can further include determining a plurality of wellness (and/or disease treatment) recommendations for the person based on the epigenetic data and the wellness goals for the person, wherein providing the indication of the one or more wellness recommendations includes ranking the plurality of wellness (and/or disease treatment) recommendations for the person based on the epigenetic data and the wellness goals for the person.

The method can further include ordering the plurality of wellness (and/or disease treatment) recommendations according to the ranking of the plurality of wellness recommendations, and providing a report of the wellness recommendations for the person according to the order of the plurality of wellness recommendations. Providing the indication of the one or more wellness recommendations can include generating a report that identifies the one or more wellness recommendations, and providing the wellness recommendations can include distributing a physical copy of the report or an electronic version of the report.

The method can further include determining scores in one or more wellness or disease categories, each score representing an assessment of the health of the person with respect to the wellness or disease category corresponding to the score.

Determining the one or more wellness recommendations for the person can include ascertaining one or more attributes of the person's health based on the epigenetic data. Ascertaining the one or more attributes of the person's health based on the epigenetic data can include comparing the epigenetic data of the person to a baseline epigenetic model. The one or more attributes of the person's health can include at least one of age, alcohol usage, body-mass index (BMI), mortality risk, inflammation, smoking habit, triglyceride level, high-density lipoprotein (HDL) level, stress level, or folate level. In some examples, the health attributes can be selected and tailored to a person's wellness and/or treatment goals, such as health attributes that are relate to goals to improve pre-natal, peri-natal, and/or post-natal health.

Determining the one or more wellness recommendations can include (i) determining a target condition for an attribute of the person's health based on at least one of the wellness goals of the person or biographical information for the person, and (ii) comparing a current condition for the attribute of the person's health as ascertained from the epigenetic data to the target condition.

The method can further include, after determining the one or more wellness recommendations for the person: obtaining second epigenetic data for the person, and updating the one or more wellness recommendations for the person based on the epigenetic data and the wellness goals for the person.

Identifying the wellness goals for the person can include accessing data representing the wellness goals for the person, the wellness goals for the person inputted at a computing device.

The method can further include tracking compliance with the one or more wellness recommendations for the person.

The method can further include sharing compliance data representing the tracked compliance of the person with the one or more wellness recommendations, the compliance data shared with at least one of a physician or other medical professional, an insurance provider, a personal trainer, a third party nominated by the service provider, social media acquaintances, or a motivator nominated by the person or the service provider.

Some implementations of the subject matter described herein include a method for assessing wellness of a person, the method including obtaining epigenetic data for the person; for each of a plurality of health attributes of the person, determining a current condition of the person with respect to the health attribute based on the epigenetic data; for each of one or more wellness characteristics, generating a score for the wellness characteristic based on the current condition of the person with respect to at least a subset of the plurality of health attributes; and providing an indication of the scores for the one or more wellness characteristics.

These and other implementations can optionally include one or more of the following features.

The epigenetic data can describe a methylation pattern in DNA of the person.

The DNA can be extracted from any biological tissue sample from the person, the biological sample including saliva, blood, or hair.

The epigenetic data can describe one or more epi-alleles in DNA of the person.

The method can further include using at least one of genetic data or biographical information of the person to determine the current condition of the person with respect to at least one of the plurality of health attributes of the person.

The method can further include generating scores for a plurality of wellness characteristics, wherein the plurality of wellness characteristics include at least one of a nutrition characteristic, a fitness characteristic, an environment characteristic, an aging characteristic, or a mindfulness characteristic.

The plurality of wellness characteristics can include the nutrition characteristic, the plurality of health attributes can include at least one of high-density lipoprotein (HDL) level, triglyceride level, folate level, or body-mass index (BMI) of the person, and generating the score for the nutrition characteristic can include generating the score based on at least one of the HDL level, triglyceride level, folate level, or BMI of the person.

The plurality of wellness characteristics can include the fitness characteristic, the plurality of health attributes can include at least one of CRP, body-mass index (BMI), MRT, or triglyceride level of the person, and generating the score for the fitness characteristic can include generating the score based on at least one of CRP, BMI, MRT, or triglyceride level of the person.

The plurality of wellness characteristics can include the mindfulness characteristic, the plurality of health attributes can include at least one of CRP, body-mass index (BMI), or MRT of the person, and generating the score for the mindfulness characteristic can include generating the score based on at least one of the CRP, BMI, or MRT of the person.

The one or more wellness characteristics can include an aging characteristic.

The one or more wellness characteristics can include an environmental characteristic, and the plurality of health attributes of the person can include at least one of a smoking attribute, alcohol usage attribute, or air pollution attribute, and generating the score for the environmental characteristic can include generating the score based on at least one of the smoking attribute, the alcohol usage attribute, or the air pollution attribute.

The method can further include determining a composite score that reflects an overall wellness of the person, the composite score determined based on a combination of scores for a plurality of wellness characteristics.

Providing the indication of the scores can include presenting the scores in a user interface of an application on a mobile computing device.

For each of the plurality of health attributes of the person, determining the current condition of the person with respect to the health attribute comprises comparing the epigenetic data of the person to a baseline epigenetic model for the health attribute.

For each of one or more wellness characteristics, generating the score for the wellness characteristic can include normalizing the score to scale the score to a pre-defined range.

The method can further include generating wellness recommendations for the person based on the scores for the one or more wellness characteristics.

Some implementations of the subject matter disclosed herein include a computer-implemented method. The method can include generating a wellness recommendation for a first person based on epigenetic data for the first person. A computing system can transmit, to a computing device of the first person, a description of the wellness recommendation for the first person. The computing system receives an indication of an activity performed by the first person in accordance with the wellness recommendation. The computing system identifies a set of motivators associated with the first person, and distributes to corresponding computing devices of the set of motivators the indication of the activity performed by the first person in accordance with the wellness recommendation. The computing system can include one or more computer in one or more locations.

These and other implementations can optionally include one or more of the following features.

The epigenetic data can describe a methylation pattern in DNA of the person.

The DNA can be extracted from a biological sample from the person, the biological sample comprising saliva, blood, or hair.

The epigenetic data can describe one or more epi-alleles in DNA of the person.

The wellness recommendation can be generated further based on a wellness goal of the first person.

The wellness recommendation can include a nutritional recommendation determined at least in part based on the epigenetic data for the first person, and the indication of the activity performed by the first person can include information about a nutrient or a food consumed by the first person.

The wellness recommendation can include a fitness recommendation determined at least in part based on the epigenetic data for the first person, and the indication of the activity performed by the first person can include information about a fitness activity performed by the first person.

The wellness recommendation can include a mindfulness recommendation determined at least in part based on the epigenetic data for the first person, and the indication of the activity performed by the first person can include information about a mindfulness activity performed by the first person.

The method can further include (i) receiving, by the computing system, a request from the first person to nominate at least some of the motivators associated with the first person, and (ii) in response to receiving the request from the first person, registering the at least some of the motivators named in the request as motivators associated with the first person.

The method can further include receiving, by the computing system, an accolade from a first motivator of the set of motivators, the accolade representing the first motivator's recognition of the activity performed by the first person in accordance with the wellness recommendation. In response to receiving the accolade from the first motivator, the computing system can provide, to the computing device of the first person, an indication of the accolade for presentation to the first person.

In response to receiving the accolade from the first motivator, the computing system can update a motivation log for the first person. The motivation log may be configured to store data about accolades awarded to the first person by each motivator of the set of motivators associated with the first person over a period of time. The motivation log, and other logs disclosed herein, may be implemented in some examples as a data structure stored on one or more memories.

The motivation log can be configured to store a count of accolades awarded to the first person over the period of time, or a value that represents a total number of points associated with accolades awarded to the first person over the period of time.

The computing system can be configured to award the first person with a benefit when the count of accolades awarded to the first person over the period of time meets a threshold or when the value that represents the total number of points associated with accolades awarded to the first person over the period of time meets a threshold.

The motivation log can track accolades awarded with respect to different activities, wellness recommendations, or categories of wellness recommendations separately from each other.

The method can further include, in response to receiving the indication of the activity performed by the first person in accordance with the wellness recommendation, registering the performance of the activity in an activity log for the first person.

Some implementations of the subject matter disclosed herein include a computer-implemented method. The method can include actions for receiving, by a computing device, data representing a wellness recommendation for a user of the computing device, the wellness recommendation generated based at least in part on epigenetic data for the user; identifying an activity performed by the user in accordance with the wellness recommendation; providing an indication of the activity performed by the user in accordance with the wellness recommendation to a set of motivators associated with the user; receiving, by the computing device, indications of accolades that motivators from the set of motivators have awarded to the user; and presenting, by the computing device, information about accolades that the motivators have awarded to the user.

These and other implementations can optionally include one or more of the following features.

The computing device can present, within a user interface, metrics that represent measurements of the user's health in a plurality of categories. The plurality of categories can be selected from a group comprising nutrition, fitness, mindfulness, and age.

The method can further include presenting, by the computing device, a newsfeed containing content curated based on at least one of wellness goals of the user or measurements of the user's health, the measurements based on the epigenetic data for the user.

Other aspects of the subject matter disclosed herein include computer-readable media and computing systems. The computer-readable media, which can have a non-transitory character, can be encoded so as to store instructions that, when executed by data processing apparatus (e.g., one or more processors), cause the data processing apparatus to perform operations according to any of the methods and processes disclosed herein. In yet other aspects, a computing system can include both the data processing apparatus and the computer-readable media. The computer-readable media can be encoded so as to store instructions that, when executed by the data processing apparatus, cause the data processing apparatus to perform operations according to any of the methods and processes disclosed herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other reference mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an example environment for epigenetic testing and assessing health or wellness conditions based on epigenetic data.

FIG. 2 depicts an example model methylation pattern for an epi-allele and a set of methylation patterns appearing on multiple instances of a DNA sequence of an individual.

FIG. 3 is a flowchart of an example process for generating wellness recommendations from epigenetic data, wellness goals, and optionally, biographical and genetic data.

FIG. 4 depicts inputs and outputs to a recommendation and scoring engine for generating wellness recommendations and health or wellness assessments.

FIG. 5 depicts a representation of example logic for generating wellness recommendations from epigenetic data, biographical information, and stated wellness goals.

FIG. 6 depicts a representation of example wellness recommendations generated for four customers based on epigenetic data, biographical information, and ranked wellness goals.

FIG. 7 is a flowchart of an example process for generating wellness scores representing corresponding wellness characteristics of a person based on epigenetic data.

FIG. 8 depicts an example report showing wellness scores for a set of wellness characteristics in different categories, and corresponding comments and actions recommended to the customer to improve wellness in each category.

FIG. 9 is a flowchart of an example process for implementing a motivators-based social network to facilitate accountability in improving epigenetic-based health markers.

FIG. 10 is a flowchart of an example process of actions performed by a user's computing device to facilitate compliance with wellness recommendations.

FIG. 11 depicts representations of example user interactions with a user interface on a computing device to access different screens showing epigenetics-based health assessments, and corresponding interpretations of the assessments and actions to improve the assessments in each of multiple categories.

FIG. 12 depicts a detailed view of a user interface in a wellness application for presenting health assessments and wellness recommendations to a user.

FIG. 13 depicts example user interface screens in a wellness application.

FIG. 14 depicts an example data structure holding customer information that may be employed by an epigenetics-based wellness service provider.

FIG. 15 shows an example of a computing device and a mobile computing device that can be used to implement at least some of the techniques described herein.

The details of one or more implementations are set forth in the accompanying drawings and the description below. Other features, objects, and advantages will be apparent from the description and drawings, and from the claims.

### DETAILED DESCRIPTION

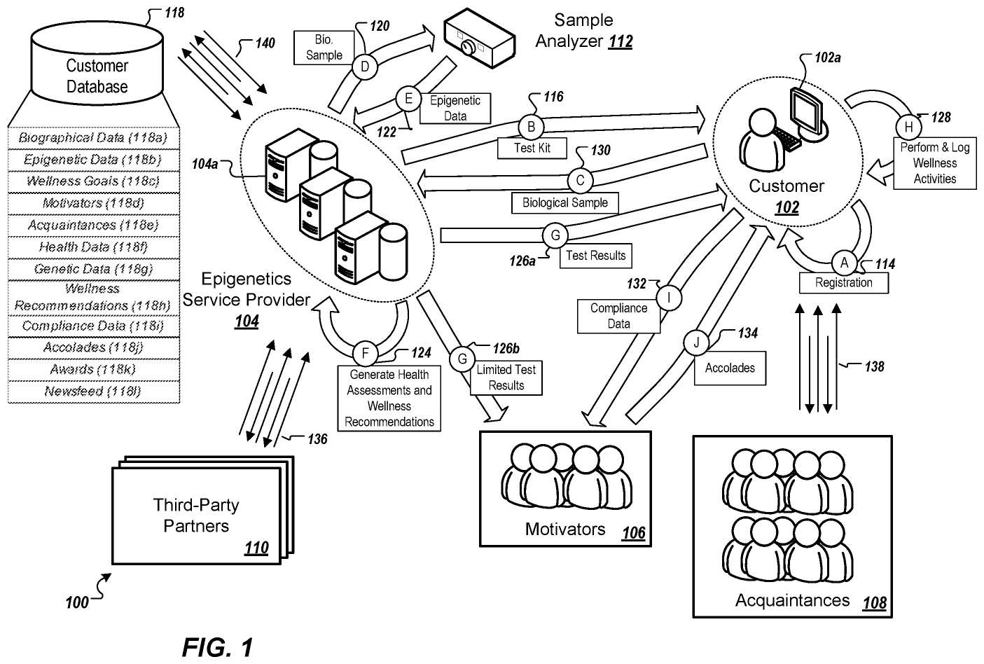

FIG. 1 shows an example environment 100 for epigenetic testing and assessing health or wellness conditions based on epigenetic data. In general, a customer 102 interacts with an epigenetics service provider 104 to obtain epigenetic test results, health assessments, and wellness recommendations based on the test results. The customer 102 can access information and services from the service provider 104 through a wellness application installed on the customer's computing device 102a. Additionally, the wellness application provides information and services to the customer 102 to facilitate compliance with the wellness recommendations. In some implementations, the wellness application maintains a compliance library in which the customer 102 registers activities performed in furtherance of one or more wellness recommendations. Records from the compliance library can be shared with a group of motivators 106, e.g., persons or other entities that the customer 102 has nominated to provide support and accountability in the customer's 102 effort to improve wellness and comply with wellness recommendations. The motivators 106 can send encouraging messages and accolades to the customer 102 for engaging in activities consistent with the wellness recommendations or other healthy lifestyle choices. The wellness application thus invokes a small social network between the customer 102 and motivators 106, and the customer 102 may improve compliance with the wellness recommendations due to a sense of accountability to the motivators 106 in his or her network.

In more detail, at stage A (114), the customer 102 determines that he/she desires to subscribe to the service provider's epigenetics-based health assessment and tracking service. The customer 102 registers with the service provider 104 by completing a survey describing necessary information about the customer 102 for the service. Although the survey may be completed by paper and mailed to the service provider 104, in a preferred embodiment, the customer 102 accesses, completes, and submits the survey online through computing device 102a (e.g., a desktop computer, laptop computer, tablet computing device, smartphone, or voice-based virtual assistant). The survey may solicit various information from the customer 102 such as biographical information (e.g., age, gender, ethnicity, height, weight, body-mass index (BMI)), behavioral information (e.g., how physically active the customer is, how often the customer drinks alcohol or smokes tobacco, sleep habits of the customer, fitness activities), and information about the customer's wellness goals (e.g., indications of how strongly the customer desires to live longer, have more energy, be happier, sleep better, increase muscle tone, improve cardiovascular fitness, reduce back or joint pain, improve nutrition, lose weight, or quit or reduce smoking or drinking).

The survey/registration information is submitted to the service provider 104. In some implementations, communications between the service provider 104, customer 102, and other parties (e.g., third-party partners 110, motivators 106, and acquaintances 108) can occur over a communications network such as the internet. For example, customer's computer 102a may transmit the survey/registration information to a computing system 104a of the service provider 104. At stage B (116), the service provider returns a test kit to the customer 102, e.g., via a postal service. The test kit may include a unique kit ID number, which the customer 102 can enter at computer 102a to associate the test kit with his/her account with the service provider 104. The test kit includes items adapted to facilitate the collection of biological samples from the customer 102 from which epigenetic information can be derived. In some implementations, the biological sample is saliva. In other implementations, the biological sample is a urine, hair, and/or blood sample. In general, any biological sample that can be adequately preserved and from which the customer's DNA can be analyzed to reliably determine epigenetic information for the customer 102 may suitable. At stage C (130), the customer 102 returns the test kit with one or more biological samples to the service provider 104.

Upon receiving the biological sample, the service provider 104 analyzes the sample to determine information about the customer's epigenetics. The analysis of the sample is represented in FIG. 1 by the provision of the sample at stage D (120) to a sample analyzer 112, and the return of epigenetic data to the service provider 104 at stage E (122) as a result. Any suitable technique as known in the field for determining methylation patterns and/or other epigenetic modifications may be employed, such as polymerase chain reaction (PCR), enrichment via CRISPR CAS9, hybridization with target oligonucleotides, microarray analysis, epi-chip, and others. Different techniques may be chosen based on factors such as cost per test, time to completion, accuracy, reliability, and test size or resolution (e.g., number of methylation sites analyzed). In some implementations, different tests may be employed for different customer service levels. For example, customers may opt to pay for a more expensive but more comprehensive and/or accurate test, or opt to pay for a less expensive but less comprehensive and/or accurate test. Moreover, although the service provider 104 in this example is shown as the entity that performs the testing on the biological sample, in other implementations the service provider 104 may contract with a third-party partner 110 to carry out testing or the customer 102 may perform testing locally, e.g., using inexpensive home-based analyzers such as the SMIDGION from OXFORD NANPORE TECHNOLOGIES.

At stage F (124), the service provider 104 generates health assessments and wellness recommendations for the customer 102. The health assessments and wellness recommendations are determined based on the test results, i.e., epigenetic data, and the customer's stated wellness goals as indicated in the survey. In some implementations, health assessments and wellness recommendations include assessments and recommendations across several wellness categories, including fitness, nutrition, and mindfulness. Mindfulness generally refers to a person's mental health, and can provide a measure, for example, of a level of stress or relaxation of the person or a measure of other attributes related to the person's parasympathetic nervous system. The health assessments can include wellness scores that numerically represent the customer's level of wellness within each of the categories. In some implementations, the health assessments and wellness scores are normalized based on biographical information for the user such as age and gender such that the assessment/score reflects how much better or worse the customer's wellness is relative to others within the same age and gender demographic as the customer 102 (or relative to a target condition for the customer's age and gender demographic). The wellness recommendations may include personalized recommendations for actions that the customer 102 can take to improve wellness in one or more categories (e.g., fitness, nutrition, mindfulness) and achieve the customer's stated wellness goals.

The service provider 104 may share the epigenetics test results, health assessments and/or wellness recommendations with one or more authorized parties. As shown in FIG. 1, the service provider's computing system 104a transmits the test results, health assessments, and/or wellness recommendations to accounts or devices of both the customer 102 (stage G (126a)) and the set of motivators 106 associated with the customer 102 (stage G (126b)). To respect the customer's privacy interests, the service provider 104 may only share information about the customer 104 with parties that the customer 102 has specifically authorized to receive such information. For example, the customer 102 may personally nominate each motivator 106 so that the customer 102 trusts them with receipt of the information. In some implementations, different parties may be authorized to receive different types of levels of information about the customer 102. For instance, the service provider 104 may provide a detailed report of the patient's epigenetic test results to a physician or other healthcare provider identified by the customer 102. The customer 102 may also receive the detailed report, or may receive a slightly less detailed report tailored to the interests and level of knowledge of the customer 102. The motivators 106 and other third parties may receive the least detailed report. For example, the motivators 106 may only receive a composite score representing an overall health assessment of the customer 102, category scores representing health assessments in one or more categories (e.g., mindfulness, fitness, nutrition, environment, aging), and/or wellness recommendations for the customer 102. In some implementations, motivators 106 may be algorithmically or randomly assigned to the customer 102 (e.g., if random assignments are authorized by the customer 102), and the motivators 106 may not receive any identifying information about the customer 102 that could reveal the true identity of the customer 102. All information about the customer 102 in such instances may be anonymized to the motivators 106. One or more of the motivators 106 may be non-human entities such as bots that use artificial intelligence and computer logic to mimic the actions of real-life persons. These artificial motivators (bots) can be programmed or trained to engage in the same types of activities as a human motivator 106. For example, an artificial motivator may process information about the customer 102 such as epigenetic test results, overall health assessment information, category scores representing health assessments in one or more categories (e.g., mindfulness, fitness, nutrition, environment, aging), or any other information that would be accessible to a human motivator 106. The artificial motivator may award accolades to the customer 102, may provide positive comments and other encouragements to the customer 102, and otherwise engage with the customer 102 as if it were a real-life person.

At stage H (128), the customer 102 access his or her health assessments and wellness recommendations through a wellness application on the customer's device 102a. The customer 102 may engage in activities consistent with the recommendations from the service provider 104, such as fitness activities, nutritious eating, and yoga or meditation or other mindfulness exercises to reduce stress and improve the customer's mental well-being. The wellness application may provide a compliance diary (compliance log) for the customer 102 to track activities relevant to the customer's wellness. For example, the customer 102 may capture photographs of his/her meals with a smartphone camera (e.g., from within the wellness application on the smartphone), and the application may employ computer vision and artificial intelligence techniques to recognize specific foods in the meals, and to rate the meal's nutritional value. Alternatively, the customer 102 use a keyboard or voice dictation to describe meals or other activities. The customer 102 may also log information about visits to the gym or other physical fitness activities, and may record information about any mindfulness or other wellness activities the customer 102 participated in. In general, the application may be programmed to automatically capture and record information about the customer's wellness activities, and/or may allow the customer 102 to manually input information about activities he or she engage in. For example, the wellness application may use location signals (e.g., GPS) to automatically recognize when the customer 102 visits a gym, and the application may also tie into services from popular fitness trackers such as FITBIT or APPLE WATCH. Likewise, the application may include mindfulness modules such as audio tracks suitable for meditative activities that can be played through the application. Playback events of the mindfulness modules may be recorded automatically in the compliance diary. Similarly, the application may suggest healthy recipes and food options personalized to the customer's wellness recommendations, and may provide an option for the user to export the recipes to create a grocery list. When a user is confirmed to have purchased meals or items consistent with nutrition recommendations, the application may log the event in a compliance diary. The application may also include an alarm clock, or interface with an external alarm clock application on the user's device, to control and set the alarm clock based on wellness recommendations to promote health sleep habits and help manage circadian rhythms. For example, if a customer's wellness recommendations indicate that the person should take an afternoon nap, sleep and wake earlier, and meditate at least once per day, the application may automatically program the alarm clock according to the recommendations. As the recommendations change or the user's wellness goals or circumstances change, the application may automatically adjust the settings of the alarm clock. Similarly, the application may perform other automated actions to facilitate compliance with wellness and/or treatment recommendations, such as automatically scheduling or initiating a scheduling process for physician visits, automatically adding recommended foods to a grocery list, or automatically adding appointments on a calendar and/or task list such as gym visits/exercise sessions and yoga sessions.

At stage I (132), data from the compliance diary is shared with the set of motivators 106 associated with the customer 102. The compliance data may be sent directly from the customer's device 102a to corresponding devices of the motivators 106, or the motivators 106 may obtain the compliance data by other means, such as from service provider 104. Compliance data may be automatically provided to the motivators 106 at periodic intervals/pre-defined times or upon the occurrence of events such as the customer's entry of a new activity into the compliance diary. Additionally or alternatively, the motivators 106 may request to obtain the customer's compliance data at any time, or the customer 102 may select times to share the compliance data with the motivators 106. Through receipt of the customer's compliance data, the motivators 106 may track the customer's lifestyle choices that bear on wellness, and may track how well the customer complies with the wellness recommendations suggested by the service provider 104. For example, the motivators 106 may be provided with pictures of meals consumed by the customer 102 over a period of time. If the meals are generally nutritious and comport with the customer's wellness recommendations, the motivators 106 may award accolades to the customer 102. An accolade can be a digital unit that represents a motivators' validation of compliant activities performed by the customer. Motivators 106 may award accolades to customers as encouragement or incentives to continue making healthy lifestyle choices and to follow through with the wellness recommendations. Motivators 106 may also send memes, private messages, or other content to the customer 102 as encouragement. At stage J (134), accolades are transmitted from computing devices of the motivators 106 to the customer 102. In an alternative embodiment, the accolades may be routed from the motivators 106 to the customer 102 via system 104a and/or other intermediaries.

In some implementations, the wellness application at the customer's device 102a, or a corresponding service at service provider system 104a, may track accolades that motivators 106 have awarded to the customer 102. For example, the wellness application may count the total number of accolades that the motivators 106 have awarded to the customer 102 over a period of time, e.g., a day, a week, a month, a quarter, or a year. When the number of awarded accolades meets a threshold value, the service provider 104 may award the customer 102 a tangible or intangible benefit for their healthy lifestyle choices and compliance with the wellness recommendations. Example benefits could include cash awards, gift cards, life insurance or medical insurance premium reductions, epigenetic testing discounts, product awards, or the like. In some cases, no benefit of value may be awarded to the customer 102, although the wellness application may present a congratulatory text or animation to the user to celebrate the achievement. The wellness application may also automatically post messages to social media accounts to provide peer recognition when the customer 102 achieves a wellness milestone (e.g., when the customer 102 is awarded a certain number of accolades, may automatically make a donation to charity, or otherwise facilitate external recognition of the customer's achievements.

In some implementations, motivators 106 may award accolades through their own installed instances of the wellness application. The wellness application may or may not impose constraints on the ability of motivators 106 to award accolades. For example, the motivators 106 may be limited to awarding no more than a pre-defined maximum number of accolades to the customer 102 over a period of time. The motivators 106 may also only be permitted to award accolades in response to indications of new or recent compliance activities performed by the customer 102. In some cases, the motivators 106 may award accolades on a per-category basis, e.g., by awarding accolades separately for each of nutrition, fitness, and mindfulness categories. The wellness application, or a corresponding service at the service provider, may then track accolades for each category, and may award benefits to the customer 102 upon meeting threshold accolade counts for each category or a combination of categories.

In some implementations, accolades may be awarded that correspond to specific activities or groups of activities logged in the compliance diary by the customer 102. To ensure that the motivators 106 award accolades to the customer 102 responsibly, the wellness application may not grant an accolade awarded to the customer 102 until at least a threshold number of motivators 106 have assented to the grant of the accolade. In this way, the system can require consensus among all or some of the motivators 106 that the customer's activity is compliant with his or her wellness recommendations before an accolade is actually granted to the customer 102. The wellness application may also be programmed to automatically alert the motivators 106 (e.g., through audible, visual, and/or haptic feedback) when new customer activity is identified for which the motivator may provide feedback (e.g., encouragement or awarding of accolades). Similarly, an alert may be provided to prompt a motivator 106 to confirm another motivator's proposed award of an accolade when consensus is required. Motivators 106 may also send encouragements and accolades to the customer 102 in other events, such as improvements in health assessments (e.g., scores) over time or when the motivator 106 is alerted to a major life event affecting the customer 102.

In some implementations, the wellness application may be programmed to implement other gamification and reward schemes that incent the customer 102 to comply with wellness and/or treatment recommendations and adopt healthy lifestyle practices. For example, the customer 102 may compete with one or more other customers to achieve one or more compliance goals. A compliance goal relates to a customer's compliance with wellness and/or treatment recommendations. For instance, a customer 102 may compete with other users to accumulate the most accolades within a given period of time, to be the first to accumulate a specified number of accolades, or to be the first to complete a wellness or treatment plan developed based on each customer's personalized wellness or treatment recommendations. The other customers may be other motivators or acquaintances of the first customer 102. The other customers with whom the first customer 102 competes may be nominated/selected by the customer 102, or automatically nominated/selected by the service provider 104 if the user elects not to choose his or her own competitors. In some examples, the system may provide competitions between groups of customers 102. Teams may be self-formed by individual customers' mutual agreements to create a team (e.g., from within a set of motivators 106) and/or teams may be formed algorithmically by the service provider's computer system or based on manual input from an agent of the service provider. Teams may compete with other teams and/or individuals to achieve one or more compliance goals, such as to be the first team to accumulate a specified number of accolades, to accumulate the most accolades within a given time period, or to be the first team to achieve a highest score that is based on each team members' individual achievements with respect to their personalized wellness and/or treatment goals. Motivators 106, for example, may award accolades to individuals on other teams and/or to the team as a unit based on the teams' compliance with wellness and/or treatment recommendations and other lifestyle and health choices made by members of a given team.

The wellness application may provide additional features in an integrated platform to help guide healthy lifestyle choices for the customer 102. For example, the wellness application may include a newsfeed that presents content items from a set of contributors related to wellness, epigenetics, and other pertinent topics. In some implementations, the newsfeed may be populated with content submitted by one or more acquaintances 108 of the customer 102. The acquaintances 108 are individuals or entities that the customer 102 has elected to follow, or that the customer 102 has connected to based on mutual agreement between the customer 102 and the acquaintance 108. Acquaintances 108 may include individuals who are also within the customer's circle of motivators 106, but motivators 106 and acquaintances 108 are generally managed separately from each other. In this way, the customer 102 may establish connections with many/unlimited number of acquaintances 108, while the motivators 106 remains a limited circle so that only individuals specifically designated as motivators 106 are eligible to receive the customer's test results, health assessments, wellness recommendations, and compliance information. For instance, acquaintances may share articles or posts on the newsfeed related to healthy living, scientific research on health-related topics, nutrition and diet information, life hacks to facilitate better lifestyle choices, fitness routines, and the like. The newsfeed may additionally or alternatively include content from other contributors selected by the service provider 104 other than acquaintances 108. For example, the newsfeed may be curated in whole or in part by the service provider 108 using human editors or algorithmic approaches to select content for inclusion in the newsfeed or block content from the newsfeed (e.g., off-topic content such as political conversations, harassment, spam, and the like). The service provider 104 may also personalize the selection of content for the customer 102 based on the epigenetics test results, wellness recommendations, wellness goals of the customer 102, biographical information of the customer 102 (e.g., age, sex, ethnicity, location), and indications of the customer's interests as reflected by the customer's prior interactions with content in the newsfeed or elsewhere in the wellness application. The wellness application may also present sponsored third-party content (e.g., ads) to the customer 102. Newsfeed content may be curated, generated by the service provider 104, third-party partners 110, motivators 106, and/or acquaintances 108, and can include targeted advertising and other content based on information about the customer 102 such as the customer's wellness and/or treatment recommendations, level of compliance with such recommendations, biographical information, epigenetic data, information about the customer's social media connections (e.g., motivators 106 and/or acquaintances 108), or a combination of these. Information shared with third parties to produce such content may be anonymized, generalized, and redacted so as to respect the privacy interests of the customer 102. The customer 102 may also authorize or deny the service provider's sharing of the customer's 102 personal data with third parties.

In some cases, the epigenetics service provider 104 maintains account information for the customer 102, and other customers, in a customer database 118. The customer database 118 may store a comprehensive overview of information for the service provider to provide relevant services to the customer 102 such as biographical data 118a, epigenetic data 118b, wellness goals 118c, identities 118d of motivators 106, identifies 118e of acquaintances 108, health assessments (e.g., scores) 118f, genetic data 118g, wellness recommendations 118h, compliance data 118i, awarded accolades 118j, awarded benefits 118k, and newsfeed content 1181.

While the preceding examples have been describes with respect to wellness recommendations to improve health attributes within categories such as fitness, nutrition, and mindfulness, the techniques can similarly be applied to assess conditions of one or more diseases of a person (e.g., customer 102) using the results of epigenetic tests on a biological sample. Assessing a disease condition may include screening for epi-alleles indicated in the person's epigenetic data that have been established to correlate with diseases such as type-II diabetes, cardiovascular diseases, cognitive decline (e.g., Alzheimer's), PTSD, certain cancers, chronic obstructive pulmonary disorder (COPD), or other diseases. In this sense, the epigenetic data may be evaluated to diagnose a disease with which the patient is presently inflicted or to predict a likelihood (e.g., a risk assessment) that the patient will develop one or more particular diseases. Based on the disease assessment, and optionally based on the patient's wellness goals, biographical information, and/or genetic data, the service provider 104 can generate treatment recommendations for the patient to treat diseases and/or to prevent the onset of certain diseases for which the epigenetic data indicates the patient has a high risk factor. The treatment recommendations may be provided along with or separately from wellness recommendations, and may be presented to the patient in similar fashion. Moreover, the wellness application may provide customized content to the user based on the patient's disease assessments and/or treatment recommendations, and if agreed to by the patient, information about the disease assessments and/or treatment recommendations can be shared with the patient's motivators so that they may be incent the patient to comply with the recommendations in a similar manner to the wellness recommendations. In some implementations, the patient may maintain multiple groups of motivators with mutually exclusive or only partially overlapping membership. For example, the patient may have a first group of motivators for wellness motivation and one or more additional groups of motivators for disease treatment motivation. In this way, the patient may be afforded maximum flexibility and control over who is authorized to receive certain information about the patient. Alternatively, the same group of motivators may be selected for both wellness and treatment recommendation compliance.

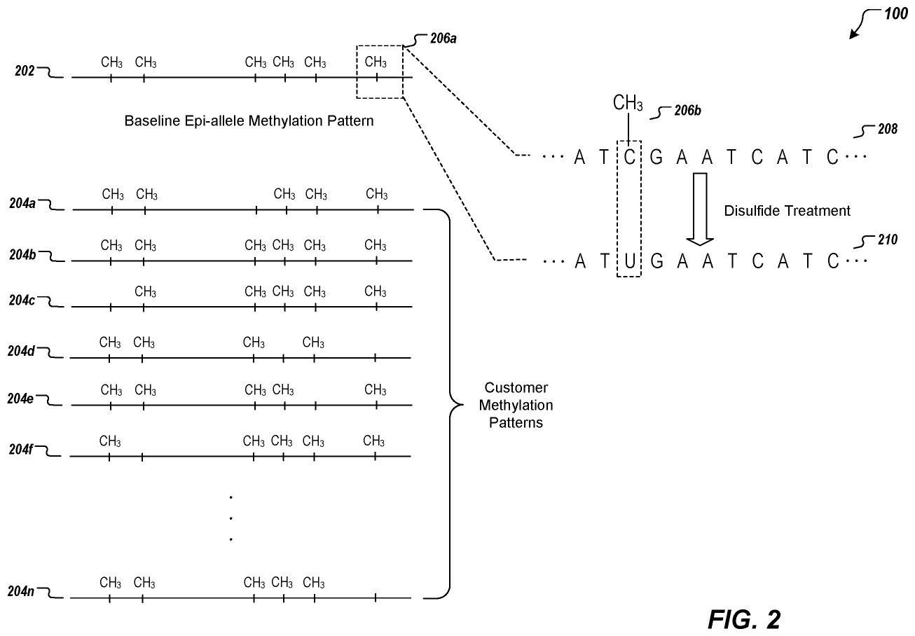

FIG. 2 depicts an example methylation pattern for an epi-allele and a set of methylation patterns appearing on multiple instances of a DNA sequence of an individual. As previously described with respect to FIG. 1, a service provider may analyze a biological sample (e.g., saliva, blood, urine, hair) of a person to derive epigenetics data that describes information about the person's health based on epigenetic patterns (e.g., methylation patterns) associated with DNA extracted from the biological sample. FIG. 2 illustrates how the analysis may be performed in some implementations. In particular, a suitable analytical technique (e.g., polymerase chain reaction or others) may be employed to analyze corresponding strands of DNA from many different cells in the biological sample. Specific sites or locations of methyl groups (CH₃) bonded to the DNA are identified from each DNA strand to determine a methylation pattern for each strand. This is illustrated by the methyl groups depicted at various sites along strands 204a-n in FIG. 2. In some implementations, methylation sites are detected by first treating the DNA (e.g., 208) with bisulfide to convert cytosine sites with attached methyl groups (e.g., site 206b) to uracil, and counting the uracil sites in the treated sequence 210 as methylated sites in the original DNA 208. To assess a condition of a person with respect to a particular health attribute (e.g., age, alcohol usage, body-mass index (BMI), mortality risk, inflammation, smoking habit, triglyceride level, high-density lipoprotein (HDL) level, or folate level), the measured methylation patterns from the customer's DNA can be compared to a baseline/model methylation pattern for the particular health attribute. The baseline/model methylation pattern, also referred to as an “epi-allele,” is a pattern that has been established as a marker for a particular phenotype or health attribute. To predict the likelihood of the customer possessing a health attribute corresponding to a particular epi-allele, the analyzer may statistically average the customer's measured methylation patterns and compare it to the baseline/model methylation pattern. For instance, a particular epi-allele may be a marker for folate deficiency, and a higher correlation between the customer's measured methylation pattern and the baseline/model methylation pattern for folate deficiency may indicate a higher likelihood of folate deficiency in the customer. Moreover, other known techniques for characterizing a person's epigenetics may be employed to determine epigenetic results from a biological sample. Methylation measurements represent one such suitable technique based on a particular epigenetic mechanism, but others may also/additionally be employed such as measuring hydroxymethylation or measuring epigenetic modifications based on other covalent modifications, RNA transcripts, microRNAs, mRNAs, sRNAs, and prions. More generally, an “epi-allele” may refer to any set of epigenetic characteristics based on one or more mechanisms that has been established as a marker for a particular phenotype or health attribute.

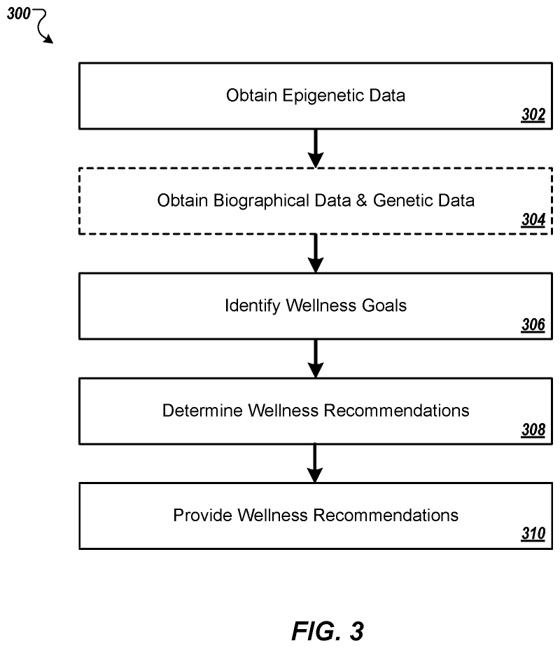

FIG. 3 is a flowchart of an example process 300 for generating wellness recommendations from epigenetic data, wellness goals, and optionally, biographical and genetic data. At stage 302, the process obtains epigenetic data for a person. The epigenetic data may indicate methylation patterns derived from analysis of the person's DNA. Additionally or alternatively, the epigenetic data may indicate measured conditions of the person with respect to one or more health attributes that have been determined based on comparison of epigenetic (e.g., methylation) patterns in the person's DNA to baseline/model epigenetic patterns (e.g., epi-alleles). Optionally, at stage 304, the process obtains biographical data (e.g., data indicating the person's age, ethnicity, location, body-mass index (BMI)) and genetic data for the person (e.g., data describing genetic scores, DNA sequences, or the presence or absence of certain genes in the person's genome). At stage 306, the process identifies wellness goals for the person. The wellness goals can include information about the person's stated desire to live longer, have more energy, be happier, sleep better, increase muscle tone, improve cardiovascular fitness, reduce back, joint, or muscle pain, improve nutrition, lose weight, reduce or quit alcoholic drinking, reduce or quit smoking, improve pre-natal health, improve pen-natal health, improve post-natal health, treat one or more particular diseases, prevent onset of one or more diseases, and improve sex life. In some implementations, goal information can include indications of how the person prioritizes certain goals over others and/or indications of the weight or import that the person attaches to each goal. For example, the goal information can include the person's ranking of all or subset of the goals from most to least important, and/or can include scores entered by the person (e.g., a score in the range 1-10) indicating the level of importance attributed to each goal.

At stage 308, the process determines one or more wellness recommendations for the person. The wellness recommendations can be based on both the epigenetic data and the person's identified goals. Thus, as between two people with the same or similar epigenetic classifications, different recommendations may be provided to each according to differences in their respective goals. In some implementations, the selection or recommendations of wellness recommendations accounts for the ranking or weight attributed to each goal. For example, a person whose goals emphasize physical fitness may be provided with recommendations aimed toward achieving the person's fitness goals while also improving overall wellness in a manner tailored based on the epigenetic data. Likewise, a person whose goals emphasize reducing back pain or pain from muscle injury may be provided with recommendations aimed toward achieving these goals. Because many people are more likely to adjust their behaviors and lifestyle to comply with a fewer number of wellness recommendations on which they can specifically focus, the system may select to return only a subset of all possible recommendations that the epigenetic analysis indicates a person's health or wellness would benefit from so that the wellness (and/or disease management) recommendations that are actually returned and presented to the person (e.g., the customer) are those that are determined to most closely align with that person's self-identified goals and/or those recommendations that are determined to address the most important or pressing issues for the person's health/wellness (e.g., based on objective criteria not specified by the user) and/or those recommendations that are predicted to have the greatest likelihood of realizing a positive and measurable impact on the person's health (e.g., recommendations that are most likely to produce a measurable change in the person's epigenetics). Each of these, and/or other, factors can be accounted for when the system selects which wellness and/or disease management recommendations to return to the user. For example, the system can determine a score for each of a plurality of candidate wellness (or disease management) recommendations across one or more wellness categories (e.g., nutrition, fitness, mindfulness) that is based on one or more of the factors disclosed herein (e.g., relevance/alignment with the user's self-identified goals, predicted likelihood of user's compliance with the recommendation, predicted likelihood of the user realizing an observable improvement in one or more health outcomes assuming full or partial compliance with the recommendation, predicted likelihood of the user realizing an observable change in epigenetics condition assuming full or partial compliance with the recommendation). The weight attributed to each factor can be based on the user's personal compliance history and/or based on others' compliance histories. The system can then rank the candidate wellness recommendations and select a pre-defined number n of the candidate recommendations to return to the user, typically the top-ranked and highest-scoring recommendations. The recommendations that fall outside of the top n recommendations are not selected and are excluded from the set returned to the user. In some implementations, a first subset of recommendations may be provided to the user. The user may then request to be presented with additional recommendations, and in response, the system may return additional ones of the plurality of candidate recommendations for the user, such as the next top n recommendations for one or more wellness categories after the initial top n recommendations that were originally returned to the user.

In some implementations, the wellness (and/or disease management/treatment) recommendations that are provided to the user/customer identify secondary activities or actions that the user/customer should engage in to improve his or her health/wellness. In contrast to primary activities or actions, secondary activities only indirectly address health/wellness needs of the user/customer as indicted by the user's epigenetics data. For example, results of an epigenetics analysis on a particular user's biologic sample may reveal that the user has a folate deficiency and is overly stressed. A primary activity or action to directly address the folate deficiency could simply be to increase folate consumption, and a wellness recommendation directed to this primary activity or action may contain an instruction for the customer/user to increase folate consumption. However, such an instruction is unlikely to be particularly helpful to the user/customer because the user/customer must then identify specific nutrition strategies on his or her own to increase folate consumption. Thus, in addition to, or alternatively to, returning recommendations describing primary activities or actions, the system may automatically return recommendations to the user that describe secondary activities or actions for improving health or wellness according to the user's epigenetics data, goals, and/or other information (e.g., biographic information, genetics information). For instance, the system may provide recommendations for specific nutritional strategies or diets that would increase the user's folate consumption, and/or may recommend specific foods, food groups, and/or recipes that contain high levels of folates for the user to consume, and a schedule for when/how much to consume. To address the user's high stress level, a primary recommendation may be to increase exercise and physical activities and to incorporate a meditation routine into the user's daily activities. A secondary recommendation may identify specific exercises and specific meditations that would aid the user's health and wellness in these area. The system may maintain a data store (e.g., a database) that maps epigenetics-based health assessments (e.g., folate deficiency, high stress) to primary wellness actions, and that maps the primary wellness actions to one or more secondary wellness actions. The system can then generate personalized wellness recommendations for the user/customer by selecting primary and/or secondary wellness actions for inclusion in the recommendations based on the any of the criteria described in this specification. In some cases, multiple secondary actions may be mapped to a single primary action, and the system may use various heuristics to select one or a subset of the multiple secondary actions to return to the user. For example, certain secondary actions may be selected based on user-specified preferences (e.g., different nutritional strategies may be employed depending on whether the user is a vegan or vegetarian), or based on indications of which secondary actions have been most successful with the user and/or other users in the past to achieve compliance and improved health outcomes.

At stage 310, the process provides the wellness recommendations to one or more interested parties, such as the user/customer, motivators associated with the user/customer, and/or a healthcare provider for the user/customer.

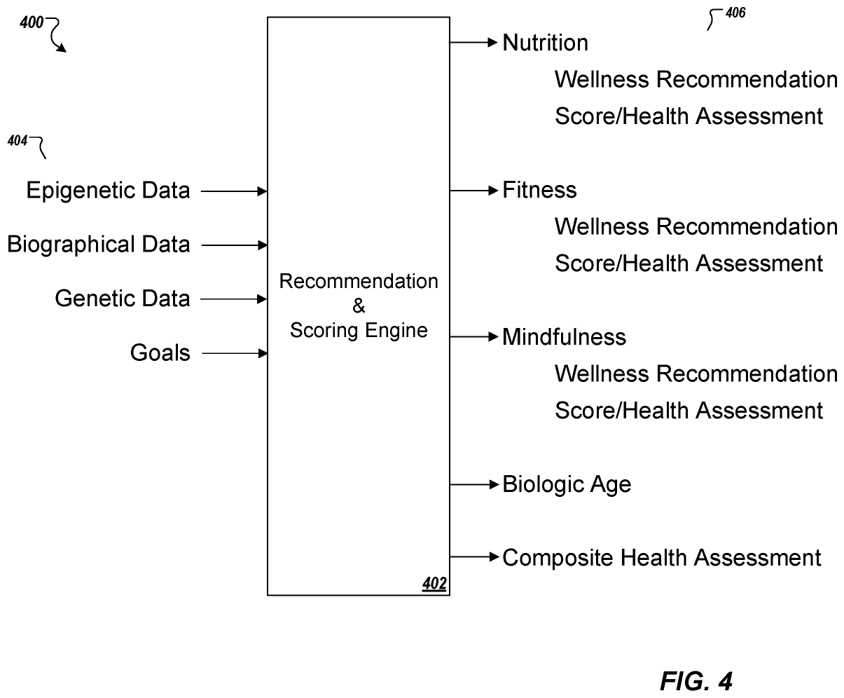

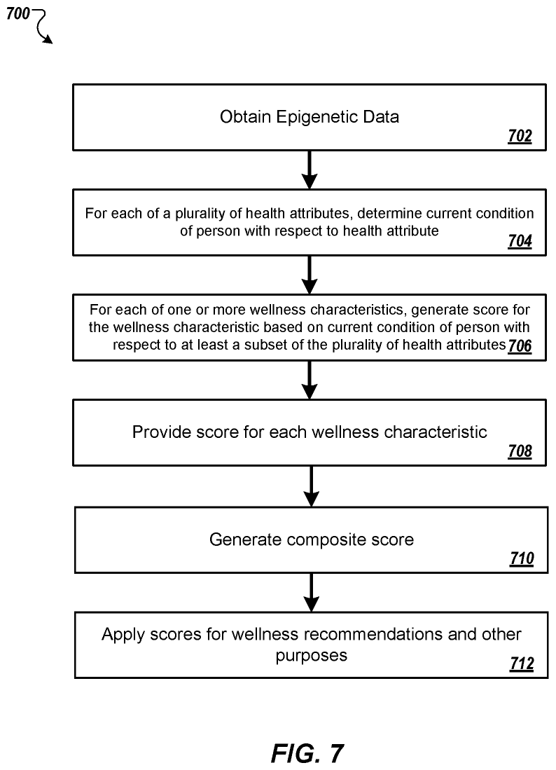

FIG. 4 is a block diagram 400 depicting inputs and outputs to a recommendation and scoring engine 402 for generating wellness recommendations and health or wellness assessments. In some implementations, the recommendation and scoring engine 402 is implemented on a computing system on one or more computers in one or more locations, e.g., service provider's system 104a. The recommendation and scoring engine 402 may be configured to carry out processes for generating health assessments (e.g., scores) and wellness recommendations, such as the processes 300 and 700 depicted in FIGS. 3 and 7, respectively. Although multiple inputs and outputs illustrated in the figure, not all of them are necessarily required. In operation, the recommendation and scoring engine 402 can process inputs 404 including epigenetic data, biographical data, genetic data, and goal information that indicates the person's/user's stated goals and absolute or relative weights of the goals. Using any of the logic and techniques disclosed herein, the engine 402 is configured to generate one or more outputs 406, including wellness recommendations for one or more wellness categories (e.g., nutrition, fitness, mindfulness) and a health assessment (e.g., a normalized score representing the health assessment) for each wellness category. The recommendation and scoring engine 402 may also generate an output indicating the biologic age of the person and/or a composite wellness assessment that reflects the person's overall wellness across multiple categories. The biologic age may be higher or lower than the person's true chronologic age, and provides a marker for a general health assessment of the person for their given age. In some implementations, the composite wellness score may be computed by averaging the wellness scores for the person across multiple categories (e.g., nutrition, fitness, mindfulness).

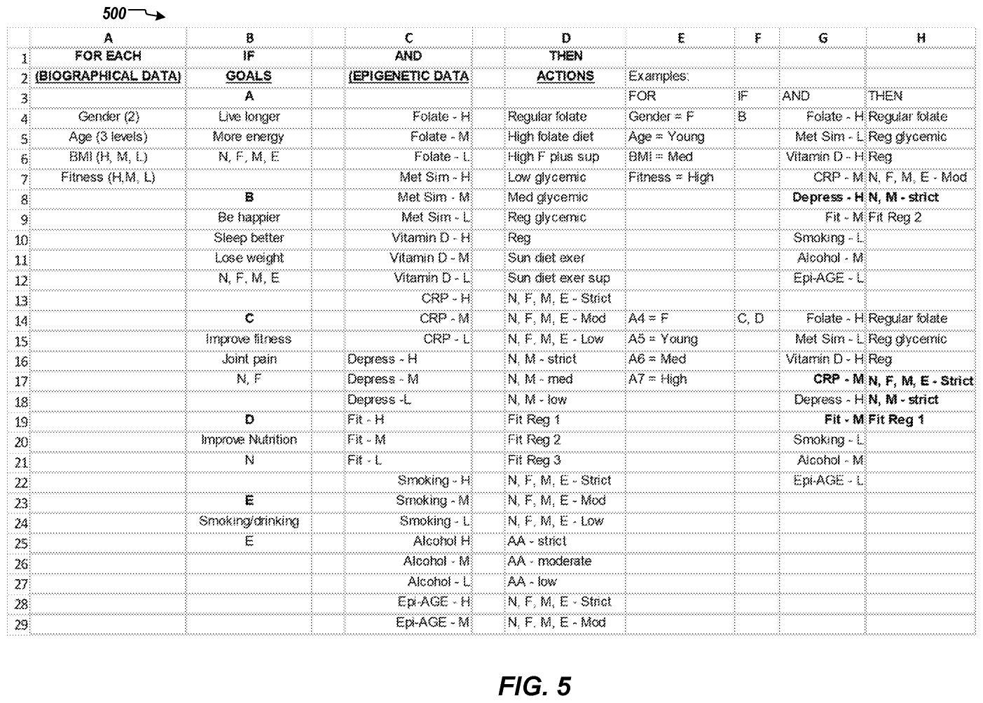

FIG. 5 depicts a representation of example logic 500 for generating wellness recommendations from epigenetic data, biographical information, and stated wellness goals. Column (A) defines several classifications for biographical information, including gender (male, female), age (young, mid, old), BMI (high, medium, low), and fitness condition (high, medium, low). Column (B) defines different goals sets such as goals set A (prioritizing goals to live longer and have more energy), goals set B (prioritizing goals to be happier, and lose weight), goals set C (prioritizing goals to improve fitness and reduce joint pain), goals set D (prioritizing goal to improve nutrition), and goals set E (prioritizing goal to reduce smoking/drinking). Column (D) defines different health attributes indicated by epigenetic data/test results, such as various folate levels, vitamin D levels, CRP, depression, fitness, and smoking/drinking impact on the person's epigenetic profile. Column (D) defines various actions that may be provided in wellness recommendations. Columns (E)-(H) provide various examples of combinations of biographical information, goals, and epigenetic data that produce different wellness recommendations.

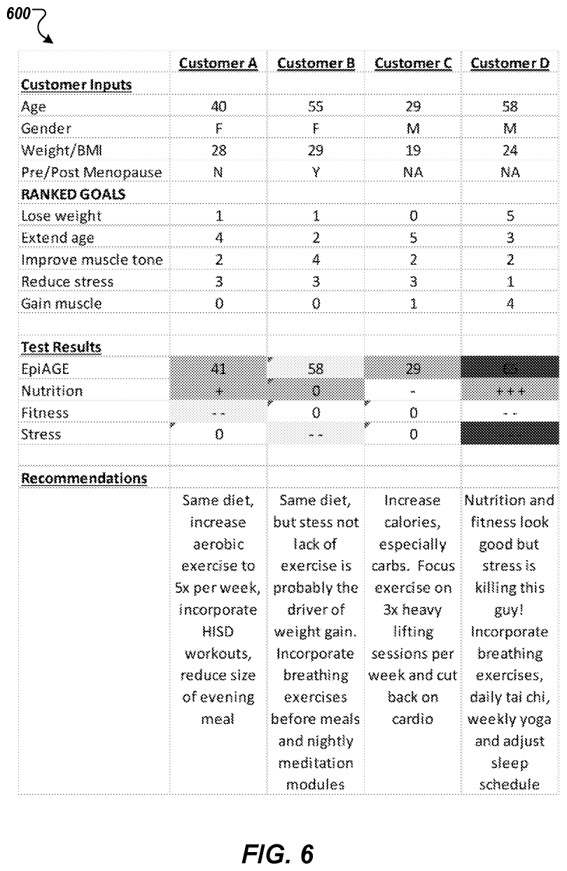

FIG. 6 depicts a representation 600 of example wellness recommendations generated for four customers based on epigenetic data, biographical information, and ranked wellness goals. Using logic similar to that disclosed in FIG. 5, a recommendation and scoring engine may process customer inputs (e.g., biographical information) such as age, gender, weight/BMI, and an indication as to whether the female customer is pre- or post-menopause, ranked goals, and epigenetic test results, to determine personalized recommendations for the customers to improve their wellness.

FIG. 7 is a flowchart of an example process 700 for generating wellness scores representing corresponding wellness characteristics of a person based on epigenetic data. At stage 702, the process obtains epigenetic data for a person. The epigenetic data may indicate methylation patterns derived from analysis of the person's DNA. Additionally or alternatively, the epigenetic data may indicate measured conditions of the person with respect to various health attributes that have been determined based on comparison of epigenetic (e.g., methylation) patterns in the person's DNA to baseline/model epigenetic patterns (e.g., epi-alleles). At stage 704, the process determines a current condition of the person with respect to each of a plurality of health attributes. The health attributes can include, for example, biologic age, alcohol usage, body-mass index (BMI), mortality risk, inflammation, smoking habit, triglyceride level, high-density lipoprotein (HDL) level, or folate level. The health attributes may already be described explicitly in the epigenetic data, or they may be derived from the epigenetic data if the epigenetic data is obtained in a raw form. At stage 706, for each one or more wellness characteristics (e.g., categories), the process generates a score for the wellness characteristic based on the current condition of the person with respect to at least a subset of the plurality of health attributes. In some implementations, the wellness characteristics include fitness, nutrition, and mindfulness, and the score for each of these characteristics is determined based on conditions for different combinations of health attributes. For example, the nutrition score may be based on HDL level, triglyceride level, micronutrient (e.g., folate) level, and BMI, while the fitness and mental stress (e.g., mindfulness) scores may be based on different combinations of attributes. In some implementations, to compute the score for a given wellness characteristic, the condition for each constituent health attribute that factors into that characteristic can be assigned a numeric weight according to whether the condition is below average, at average, or above average. The weights for each constituent health attribute for the wellness characteristic can be summed to determine a raw score for the wellness characteristic. The raw score may then be normalized and scaled according to the following formula: Normalized score=1000×(Raw Score−Minimum Possible Score)/Maximum Possible Score. The normalized score may be scaled to a range (e.g., 0-1000) that is sufficient to allow the customer to realize noticeable changes in the score from one test period to another (e.g., once a month or once every 3 or 6 months). At stage 710, the process can generate a composite score, e.g., by averaging the respective normalized scores for each of the wellness characteristics. At stage 712, the process may apply the scores to one or more practical ends. In some implementations, the scores are presented to a user in a report, e.g., in a user interface of a wellness application on the user's personal computing device. In some implementations, the scores can also be used to show trends in the person's wellness across each characteristic over time such as across two, three, four, or more testing periods. In some implementations, the recommendation and scoring engine may use the wellness scores in determining wellness recommendations for the user. For example, the recommendation and scoring engine may compare the wellness scores to the user's personalized wellness goals, and may automatically boost or discount certain goals based on which wellness characteristics the scores indicate are in need of most improvement. In some implementations, the service provider may track customers' wellness scores over time, and may use information about changes in the wellness scores over time and information about the customers' compliance with wellness recommendations to assess the efficacy of wellness recommendations, and to refine the wellness recommendations provided to customers to reflect activities that the customer can engage in that are most likely to impact overall wellness and individual wellness scores.

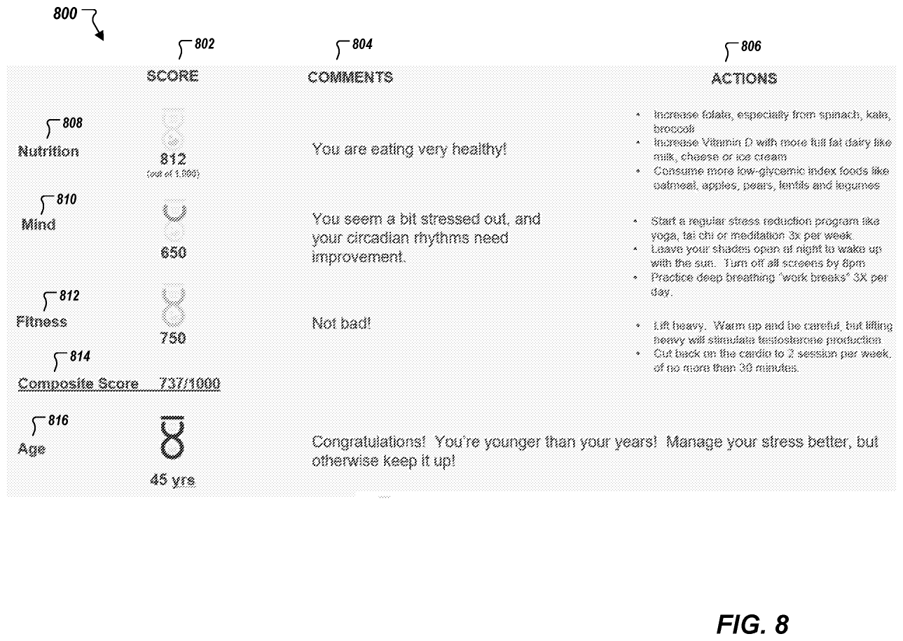

FIG. 8 depicts an example report 800 showing wellness scores 802 for a set of wellness characteristics in different categories 808-812, and corresponding comments 804 and actions 806 recommended to the customer to improve wellness in each category. The report 800 can also include a composite wellness score 814 and a biologic age 816 derived from the epigenetic data. In some implementations, the report 800 is formatted for presentation in a user interface of a wellness application on a mobile device or other personal computing device. Where the report includes multiple wellness and/or treatment recommendations, the service provider may rank the recommendations as a whole or within each category based on priority, and may present the recommendations in the report in an order according to the ranking. For example, a recommendation that requires immediate attention as pertaining to a serious health risk may be prioritized over a recommendation that relates to a less-imminent health outcome, and the higher-priority recommendation may be displayed first or otherwise more prominently than the lower-priority recommendation. Moreover, the wellness application may be configured to spread the presentation of wellness recommendations out over time, or otherwise limit the number of recommendations that are presented at one time so as to avoid overwhelming the customer. The customer 102 may be more likely to comply with the recommendations by focusing on just or a few at a time, and gradually incorporating additional recommendations into their lifestyle or routine.

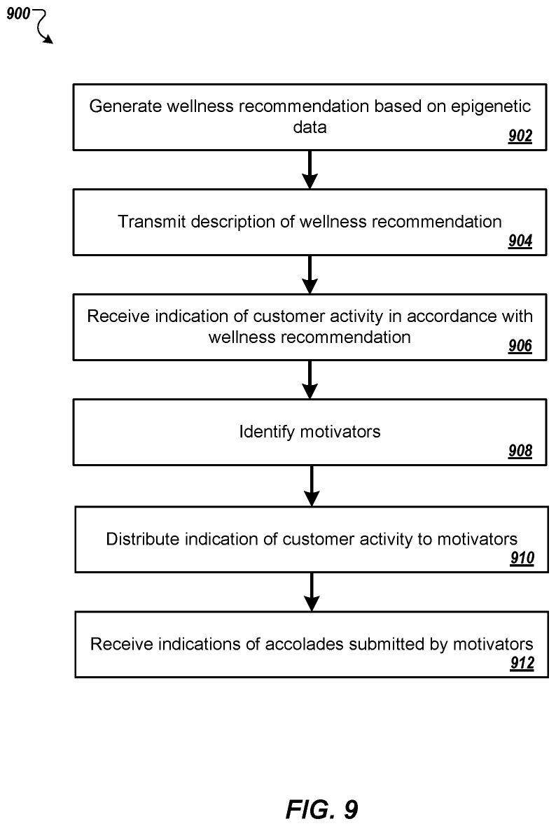

FIG. 9 is a flowchart of an example process 900 for implementing a motivators-based social network to facilitate accountability in improving epigenetic-based health markers. The process 900 can be performed by a computing system, e.g., service provider's system 104a. At stage 902, the system generates wellness recommendations for a person based on epigenetic data for that person. At stage 904, the system transmits a description of the wellness recommendation, and optionally additional information such as a lower-level report about the epigenetic test results and wellness scores for each of one or more wellness categories, to a computing device accessed by the person. At stage 906, the system receives an indication of customer activity performed in accordance with a wellness recommendation. For example, the user may log activities in a compliance diary in a wellness application at his or her personal device, and entries from the log may be provided to the service provider's system. At stage 908, the system identifies a set of motivators associated with the user. The motivators may have been previously nominated and selected by the user as individuals or entities from whom the user desires support and the provision of accountability for the user to implement lifestyle changes and choices that may improve the user's health and wellness. In some implementations, the motivators may be automatically selected by the system using computer-based logic and algorithms based on one or more factors. For example, users who are themselves customers of the service provider may be selected as motivators for a first user based on similarities in demographics, epigenetics data, wellness goals, or wellness recommendations between those users and the first user. The system may also rate motivators based on a scoring framework that assigns scores to motivators that reflect their effectiveness as motivators. The scores may be based on feedback from customers/users about the effectiveness of their motivators and/or based on objective criteria such as how frequently the motivators have previously offered encouragement to the users they have been assigned to motivate, the quality of motivations offered, and the frequency of the motivator's interaction with the service provider's ecosystem. The rating (e.g., score) for the motivator may be used as a basis for algorithmic selection of motivators for future users such that higher-rated motivators are more likely to be selected than lower-rated motivators. Motivators' ratings can also be presented to users who elect to self-nominate their motivators. In some implementations, a semi-automated process may be employed to assign motivators to users. The system may automatically identify a pool of candidate motivators for a new user, and the user may review ratings and profile information for the candidate motivators, and then nominate or select a subset of motivators from the pool to be assigned to the user.

At stage 910, the system distributes compliance information indicating customer activity to the motivators. The motivators can review the compliance information and determine whether they comport with healthy lifestyle choices consistent with the wellness recommendations provided to the user. If so, the motivators may award the user with accolades (stage 912).

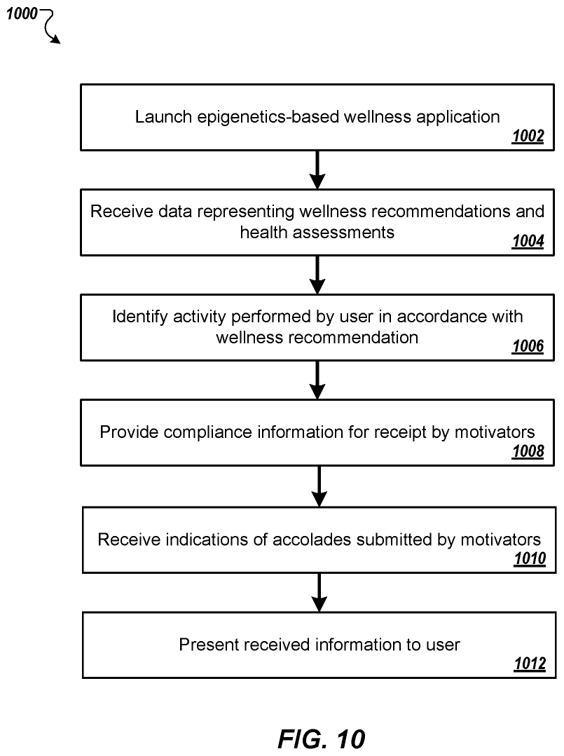

FIG. 10 is a flowchart of an example process 1000 of actions performed by a user's computing device to facilitate compliance with wellness recommendations. The user can launch an epigenetics-oriented wellness application that is either installed on the device or otherwise accessible to the device, e.g., through a cloud-based service (stage 1002). After having provided a biological sample for testing and test results having been completed, the device may receive data from the service provider representing wellness recommendations and health assessments personalized to the user (stage 1004). The device may present the information in a user interface on a screen of the device. The device may receive an indication that a user has completed an entry and made an entry in a compliance diary to reflect the same (stage 1006). The device may then forward compliance information from the user's compliance diary, or a summarized and/or anonymized version of the same, to the set of motivators associated with the user (stage 1008). The motivators may review the compliance information and, if appropriate, award the user with accolades for positive behaviors that promote wellness. Indications of awarded accolades may be received by and registered by the wellness application at the user's computer device (stage 1010). Information about accolades, newsfeeds, encouragements, wellness recommendations, wellness scores, and other information may be presented to the user through the user interface of the wellness application (stage 1012).

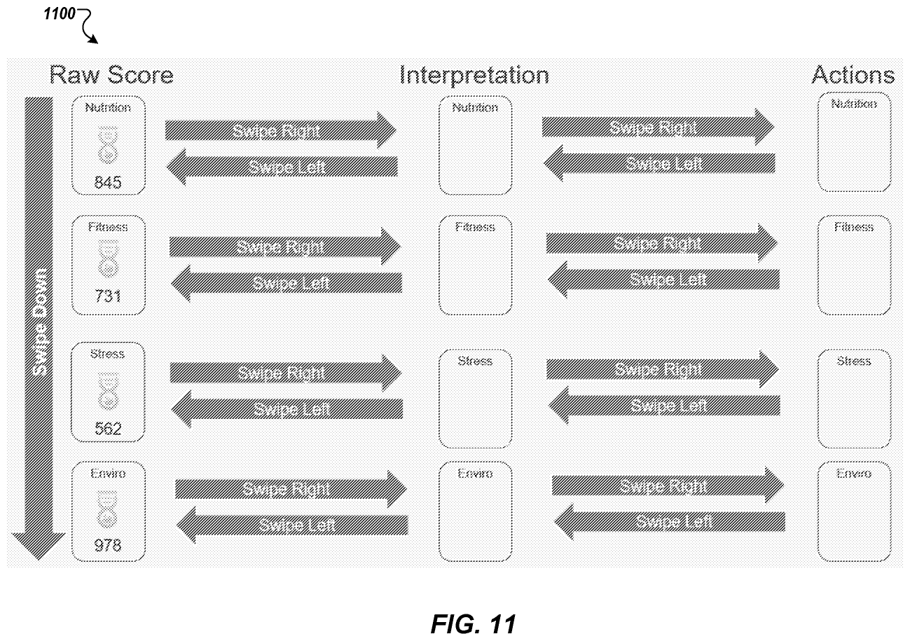

FIG. 11 depicts representations of example user interactions 1100 with a user interface on a computing device to access different screens showing epigenetics-based health assessments, and corresponding interpretations of the assessments and actions to improve the assessments in each of multiple categories. For example, a user can access a first screen that depicts the user's normalized nutrition score (e.g., 845) based on epigenetic test results. An hourglass-like symbol or other meter may be shown on the screen which is filled to a level corresponding to the user's wellness score. The user may swipe right to obtain further details (e.g., an interpretation) of the score, and then swipe right again to access a screen that includes text and/or graphics describing wellness recommendations (e.g., actions) that the user can take to improve wellness in the corresponding area (e.g., nutrition). The user may also swipe up or down to transition between assessments/scores and interpretations/actions in different categories (e.g., nutrition, fitness stress/mindfulness, and environment).

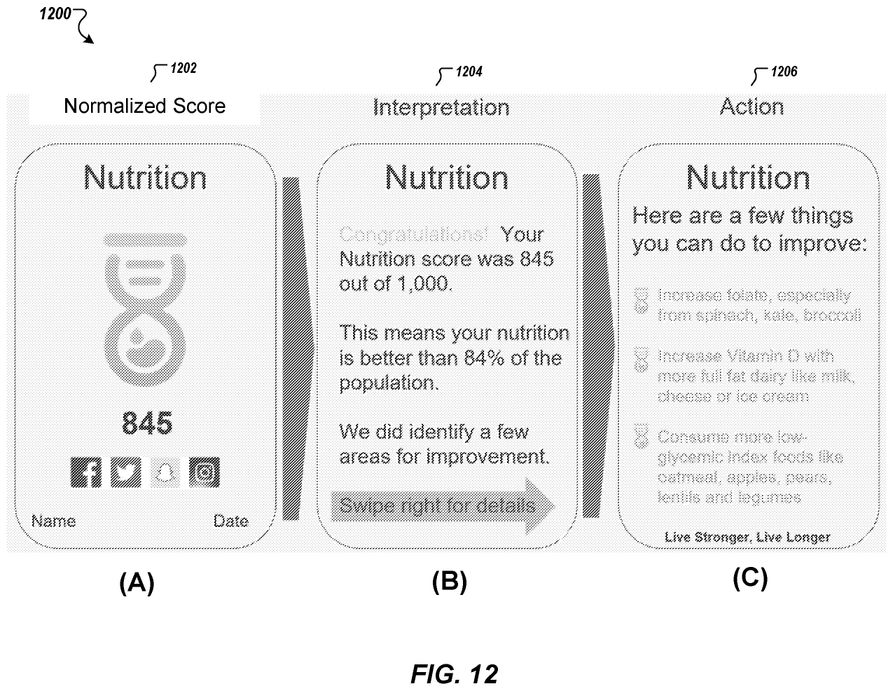

FIG. 12 depicts a detailed view of a user interface 1200 in a wellness application for presenting health assessments and wellness recommendations to a user. As shown, the user can swipe between screens presenting a normalized score 1202 (e.g., for a nutrition characteristic), an interpretation of the score 1204, and an action (e.g., wellness recommendation) 1206. From one or more of the screens, the user may also select control elements to share the score on social media platforms such as FACEBOOK, TWITTER, INSTAGRAM, or WHATSAPP.

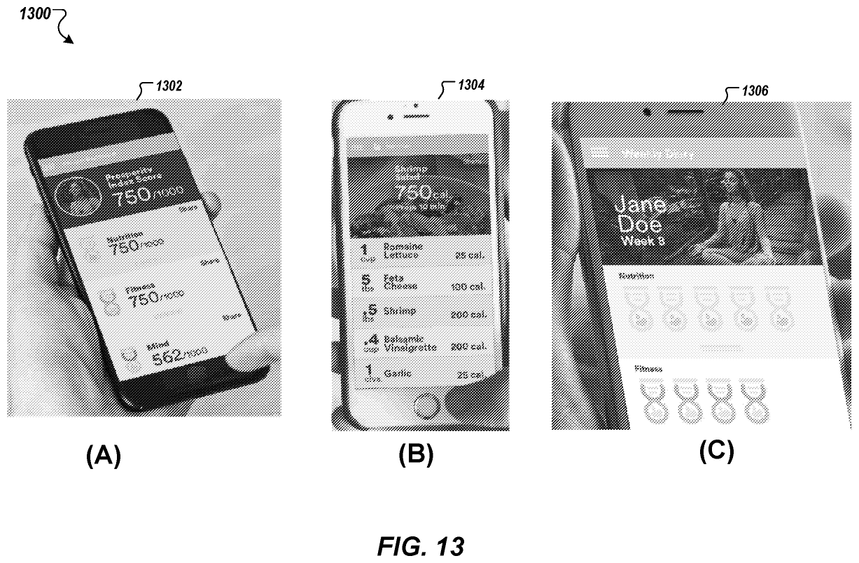

FIG. 13 depicts example user interface screens in a wellness application. Screen A (1302) shows a dashboard with a composite index score presented atop, and normalized wellness scores for a plurality of wellness characteristics beneath the composite index score. Screen B (1304) shows a recommended recipe that can be accessed through the application as a recommended food for improving the user's consumption of folates or other micronutrients. Screen C (1306) shows a user's weekly compliance diary with indications of wellness activities performed in that week in one or more categories and accolades awarded by motivators with respect to those wellness activities.

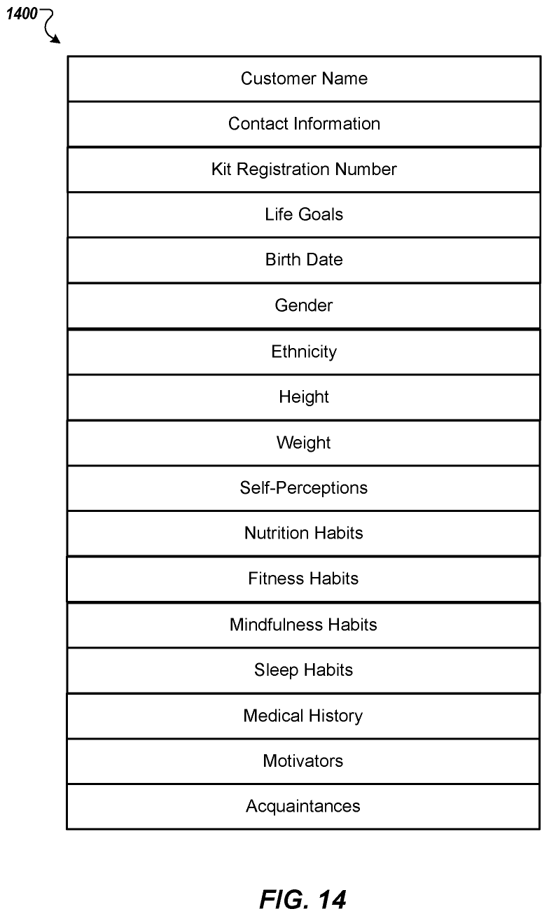

FIG. 14 depicts an example data structure 1400 holding customer information that may be employed by an epigenetics-based wellness service provider. In some implementations, the data structure 1400 is stored in a database at a computing system of the service provider, e.g., system 104a.

### Examples of First Illustrative Embodiment

A1. A method for providing personalized wellness recommendations, comprising:

obtaining epigenetic data for a person;

identifying wellness goals for the person;

determining one or more wellness recommendations for the person based on the epigenetic data and the wellness goals for the person; and

providing an indication of the one or more wellness recommendations.

A2. The method of A1, further comprising:

obtaining genetic data for the person; and

determining the one or more wellness recommendations for the person further based on the genetic data.

A3. The method of A2, wherein the genetic data comprises polygenic scores.
A4. The methods of any of A1-A3, further comprising:
obtaining biographical information for the person; and determining the one or more wellness recommendations for the person further based on the biographical information.
   A5. The method of A4, wherein the biographical information comprises information indicating at least one of an age, gender, ethnicity, height, weight, body-mass index (BMI), waist circumference, wrist circumference, blood glucose monitoring data, fitness tracking data, or medical history of the person.
   A6. The methods of any of A1-A5, wherein the epigenetic data describes a methylation pattern in DNA of the person.
   A7. The method of A6, wherein the DNA is extracted from a biological sample from the person, the biological sample comprising saliva, blood, or hair.
   A8. The methods of any of A1-A7, wherein the epigenetic data describes one or more epi-alleles in DNA of the person.
   A9. The methods of any of A1-A8, wherein the wellness goals are selected from a group comprising goals to live longer, have more energy, be happier, sleep better, increase muscle tone, improve cardiovascular fitness, reduce back, joint, or muscle pain, improve nutrition, lose weight, reduce or quit alcoholic drinking, and reduce or quit smoking.
   A10. The methods of any of A1-A9, further comprising:

assigning weights to the wellness goals; and

determining the wellness recommendations for the person using the weights for the wellness goals.

A11. The methods of any of A1-A10, wherein determining the wellness recommendations for the person comprises determining recommendations for the person in one or more wellness categories.

A12. The methods of any of A1-A11, further comprising selecting the one or more wellness categories from a plurality of wellness categories based on at least one of the wellness goals for the person, the epigenetic data for the person, biographical information for the person, or genetic data for the person.

A13. The methods of any of A1-A12, wherein the wellness categories include at least one of a nutrition category, a fitness category, or a mindfulness category.
A14. The methods of any of A1-A13, wherein the wellness categories include a nutrition category, and the wellness recommendations comprise a recommendation for a suggested nutrient, food, or food group for the person to consume to improve nutrition.
A15. The methods of any of A1-A14, wherein the wellness categories include a fitness category, and the wellness recommendations comprise a recommendation for a suggested fitness activity or type of fitness activity for the person to engage in to improve fitness.
A16. The methods of any of A1-A15, wherein the wellness categories include a mindfulness category, and the recommendations comprise a recommendation for a suggested mindfulness activity or mindfulness routine for the person to engage in to improve mindfulness.
A17. The methods of any of A1-A16, further comprising determining a plurality of wellness recommendations for the person based on the epigenetic data and the wellness goals for the person, wherein providing the indication of the one or more wellness recommendations comprises ranking the plurality of wellness recommendations for the person based on the epigenetic data and the wellness goals for the person.
A18. The methods of any of A1-A17, further comprising:

ordering the plurality of wellness recommendations according to the ranking of the plurality of wellness recommendations; and

providing a report of the wellness recommendations for the person according to the order of the plurality of wellness recommendations.

A19. The methods of any of A1-A18, wherein providing the indication of the one or more wellness recommendations comprises generating a report that identifies the one or more wellness recommendations, and providing the wellness recommendations comprises distributing a physical copy of the report an electronic version of the report.
A20. The methods of any of A1-A19, further comprising determining scores in one or more wellness categories, each score representing an assessment of the health of the person with respect to the wellness category corresponding to the score.
A21. The methods of any of A1-A20, wherein determining the one or more wellness recommendations for the person comprises ascertaining one or more attributes of the person's health based on the epigenetic data.
A22. The method of A21, wherein ascertaining the one or more attributes of the person's health based on the epigenetic data comprises comparing the epigenetic data of the person to a baseline epigenetic model.
A23. The method of A21, wherein the one or more attributes of the person's health comprise at least one of age, alcohol usage, body-mass index (BMI), mortality risk, inflammation, smoking habit, triglyceride level, high-density lipoprotein (HDL) level, or folate level.
A24. The methods of any of A1-A23, wherein determining the one or more wellness recommendations comprises:

determining a target condition for an attribute of the person's health based on at least one of the wellness goals of the person or biographical information for the person; and

comparing a current condition for the attribute of the person's health as ascertained from the epigenetic data to the target condition.

A25. The methods of any of A1-A24, further comprising, after determining the one or more wellness recommendations for the person:

obtaining second epigenetic data for the person; and

updating the one or more wellness recommendations for the person based on the epigenetic data and the wellness goals for the person.

A26. The methods of any of A1-A25, wherein identifying the wellness goals for the person comprises accessing data representing the wellness goals for the person, the wellness goals for the person inputted at a computing device.
A27. The methods of any of A1-A26, further comprising tracking compliance with the one or more wellness recommendations for the person.
A28. The method of A27, further comprising sharing compliance data representing the tracked compliance of the person with the one or more wellness recommendations, the compliance data shared with at least one of a physician, an insurance provider, or motivator nominated by the person.
A29. One or more non-transitory computer-readable media having instructions stored thereon that, when executed by one or more processors, cause the one or more processors to perform any of the methods of A1-A28.
A30. A system, comprising:

one or more processors; and

one or more computer-readable media having instructions stored thereon that, when executed by the one or more processors, cause the one or more processors to perform any of the methods of A1-A28.

### Examples of Second Illustrative Embodiment

B1. A method for assessing wellness of a person, comprising:

obtaining epigenetic data for the person;

for each of a plurality of health attributes of the person, determining a current condition of the person with respect to the health attribute based on the epigenetic data;

for each of one or more wellness characteristics, generating a score for the wellness characteristic based on the current condition of the person with respect to at least a subset of the plurality of health attributes; and providing an indication of the scores for the one or more wellness characteristics.

B2. The method of B1, wherein the epigenetic data describes a methylation pattern in DNA of the person.
B3. The method of B2, wherein the DNA is extracted from a biological sample from the person, the biological sample comprising saliva, blood, or hair.
B4. The methods of any of B1-B3, wherein the epigenetic data describes one or more epi-alleles in DNA of the person.
B5. The methods of any of B1-B4, further comprising using at least one of genetic data or biographical information of the person to determine the current condition of the person with respect to at least one of the plurality of health attributes of the person.
B6. The methods of any of B1-B5, further comprising generating scores for a plurality of wellness characteristics, wherein the plurality of wellness characteristics comprise at least one of a nutrition characteristic, a fitness characteristic, or a mindfulness characteristic.
B7. The method of B6, wherein the plurality of wellness characteristics includes the nutrition characteristic, the plurality of health attributes includes at least one of high-density lipoprotein (HDL) level, triglyceride level, folate level, or body-mass index (BMI) of the person, and generating the score for the nutrition characteristic comprises generating the score based on at least one of the HDL level, triglyceride level, folate level, or BMI of the person.
B8. The method of B6, wherein the plurality of wellness characteristics includes the fitness characteristic, the plurality of health attributes includes at least one of CRP, body-mass index (BMI), MRT, or triglyceride level of the person, and generating the score for the fitness characteristic comprises generating the score based on at least one of CRP, BMI, MRT, or triglyceride level of the person.
B9. The method of B6, wherein the plurality of wellness characteristics includes the mindfulness characteristic, the plurality of health attributes includes at least one of CRP, body-mass index (BMI), or MRT of the person, and generating the score for the mindfulness characteristic comprises generating the score based on at least one of the CRP, BMI, or MRT of the person.
B10. The methods of any of B1-B9, wherein the one or more wellness characteristics includes an aging characteristic.
B11. The methods of any of B1-B10, wherein the one or more wellness characteristics includes an environmental characteristic, and the plurality of health attributes of the person includes at least one of a smoking attribute, alcohol usage attribute, or air pollution attribute, and generating the score for the environmental characteristic comprises generating the score based on at least one of the smoking attribute, the alcohol usage attribute, or the air pollution attribute.
B12. The methods of any of B1-B11, further comprising determining a composite score that reflects an overall wellness of the person, the composite score determined based on a combination of scores for a plurality of wellness characteristics.
B13. The methods of any of B1-B12, wherein providing the indication of the scores comprises presenting the scores in a user interface of an application on a mobile computing device.
B14. The methods of any of B1-B13, wherein for each of the plurality of health attributes of the person, determining the current condition of the person with respect to the health attribute comprises comparing the epigenetic data of the person to a baseline epigenetic model for the health attribute.
B15. The methods of any of B1-B14, wherein for each of one or more wellness characteristics, generating the score for the wellness characteristic comprises normalizing the score to scale the score to a pre-defined range.
B16. The methods of any of B1-B15, further comprising generating wellness recommendations for the person based on the scores for the one or more wellness characteristics.
B17. One or more non-transitory computer-readable media having instructions stored thereon that, when executed by one or more processors, cause the one or more processors to perform any of the methods of B1-B16.
B18. A system, comprising:

one or more processors; and

one or more computer-readable media having instructions stored thereon that, when executed by the one or more processors, cause the one or more processors to perform any of the methods of B1-B16.

### Examples of Third Illustrative Embodiment

C1. A computer-implemented method, comprising:

generating a wellness recommendation for a first person based on epigenetic data for the first person;

transmitting, from a computing system and to a computing device of the first person, a description of the wellness recommendation for the first person;

receiving, by the computing system, an indication of an activity performed by the first person in accordance with the wellness recommendation;

identifying, by the computing system, a set of motivators associated with the first person; and

distributing, by the computing system and to corresponding computing devices of the set of motivators associated with the first person, the indication of the activity performed by the first person in accordance with the wellness recommendation.

C2. The computer-implemented method of C1, wherein the epigenetic data describes a methylation pattern in DNA of the person.
C3. The computer-implemented method of C2, wherein the DNA is extracted from a biological sample from the person, the biological sample comprising saliva, blood, or hair.
C4. The computer-implemented methods of any of C1-C3, wherein the epigenetic data describes one or more epi-alleles in DNA of the person.
C5. The computer-implemented methods of any of C1-C4, wherein the wellness recommendation is generated further based on a wellness goal of the first person.
C6. The computer-implemented methods of any of C1-05, wherein the wellness recommendation comprises a nutritional recommendation determined at least in part based on the epigenetic data for the first person, and the indication of the activity performed by the first person comprises information about a nutrient or a food consumed by the first person.
C7. The computer-implemented methods of any of C1-C6, wherein the wellness recommendation comprises a fitness recommendation determined at least in part based on the epigenetic data for the first person, and the indication of the activity performed by the first person comprises information about a fitness activity performed by the first person.
C8. The computer-implemented methods of any of C1-C7, wherein the wellness recommendation comprises a mindfulness recommendation determined at least in part based on the epigenetic data for the first person, and the indication of the activity performed by the first person comprises information about a mindfulness activity performed by the first person.
C9. The computer-implemented methods of any of C1-C8, further comprising:

receiving, by the computing system, a request from the first person to nominate at least some of the motivators associated with the first person; and

in response to receiving the request from the first person, registering the at least some of the motivators named in the request as motivators associated with the first person.

C10. The computer-implemented methods of any of C1-C9, further comprising receiving, by the computing system, an accolade from a first motivator of the set of motivators, the accolade representing the first motivator's recognition of the activity performed by the first person in accordance with the wellness recommendation.
C11. The computer-implemented method of C10, in response to receiving the accolade from the first motivator, providing by the computing system and to the computing device of the first person, an indication of the accolade for presentation to the first person.
C12. The computer-implemented method of C10, in response to receiving the accolade from the first motivator, updating a motivation log for the first person, the motivation log configured to store data about accolades awarded to the first person by each motivator of the set of motivators associated with the first person over a period of time.
C13. The computer-implemented method of C12, wherein the motivation log is configured to store a count of accolades awarded to the first person over the period of time or a value that represents a total number of points associated with accolades awarded to the first person over the period of time.
C14. The computer-implemented method of C12, wherein the computing system is configured to award the first person with a benefit when the count of accolades awarded to the first person over the period of time meets a threshold or when the value that represents the total number of points associated with accolades awarded to the first person over the period of time meets a threshold.
C15. The computer-implemented method of C12, wherein the motivation log tracks accolades awarded with respect to different activities, wellness recommendations, or categories of wellness recommendations separately from each other.
C16. The computer-implemented methods of any of C1-C15, further comprising in response to receiving the indication of the activity performed by the first person in accordance with the wellness recommendation, registering the performance of the activity in an activity log for the first person.
C17. One or more non-transitory computer-readable media having instructions stored thereon that, when executed by one or more processors, cause the one or more processors to perform any of the methods of C1-C16.
C18. A system, comprising:

one or more processors; and

one or more computer-readable media having instructions stored thereon that, when executed by the one or more processors, cause the one or more processors to perform any of the methods of C1-C16.

C19. A computer-implemented method, comprising:

receiving, by a computing device, data representing a wellness recommendation for a user of the computing device, the wellness recommendation generated based at least in part on epigenetic data for the user;

identifying an activity performed by the user in accordance with the wellness recommendation;

providing an indication of the activity performed by the user in accordance with the wellness recommendation to a set of motivators associated with the user;

receiving, by the computing device, indications of accolades that motivators from the set of motivators have awarded to the user; and

presenting, by the computing device, information about accolades that the motivators have awarded to the user.

C20. The computer-implemented method of C19, further comprising presenting, by the computing device and within a user interface, metrics that represent measurements of the user's health in a plurality of categories.
C21. The computer-implemented method of C20, wherein the plurality of categories are selected from a group comprising nutrition, fitness, mindfulness, and age.
C22. The computer-implemented methods of any of C19-C21, further comprising presenting, by the computing device, a newsfeed containing content curated based on at least one of wellness goals of the user or measurements of the user's health, the measurements based on the epigenetic data for the user.

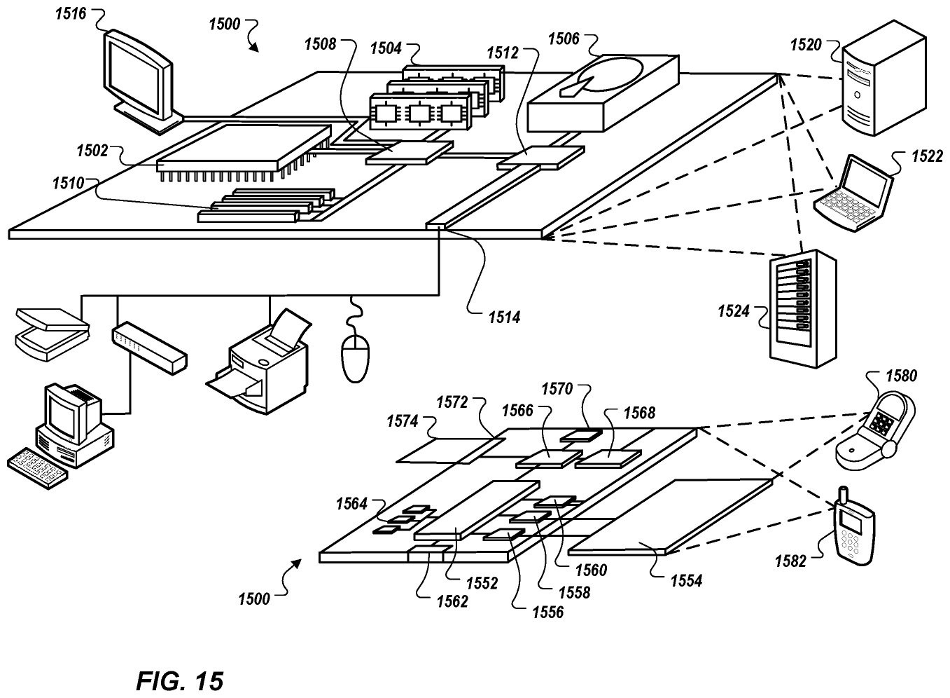

FIG. 15 shows an example of a computing device 1500 and a mobile computing device 1550 that can be used to implement the techniques described herein. The computing device 1500 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1500 includes a processor 1502, a memory 1504, a storage device 1506, a high-speed interface 1508 connecting to the memory 1504 and multiple high-speed expansion ports 1510, and a low-speed interface 1512 connecting to a low-speed expansion port 1514 and the storage device 1506. Each of the processor 1502, the memory 1504, the storage device 1506, the high-speed interface 1508, the high-speed expansion ports 1510, and the low-speed interface 1512, are interconnected using various busses, and may be mounted on a common motherboard or in other manners as appropriate. The processor 1502 can process instructions for execution within the computing device 1500, including instructions stored in the memory 1504 or on the storage device 1506 to display graphical information for a GUI on an external input/output device, such as a display 1516 coupled to the high-speed interface 1508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 1504 stores information within the computing device 1500. In some implementations, the memory 1504 is a volatile memory unit or units. In some implementations, the memory 1504 is a non-volatile memory unit or units. The memory 1504 may also be another form of computer-readable medium, such as a magnetic or optical disk.

The storage device 1506 is capable of providing mass storage for the computing device 1500. In some implementations, the storage device 1506 may be or contain a computer-readable medium, such as a floppy disk device, a hard disk device, an optical disk device, or a tape device, a flash memory or other similar solid state memory device, or an array of devices, including devices in a storage area network or other configurations. The computer program product may also contain instructions that, when executed, perform one or more methods, such as those described above. The computer program product can also be tangibly embodied in a computer- or machine-readable medium, such as the memory 1504, the storage device 1506, or memory on the processor 1502.

The high-speed interface 1508 manages bandwidth-intensive operations for the computing device 1500, while the low-speed interface 1512 manages lower bandwidth-intensive operations. Such allocation of functions is exemplary only. In some implementations, the high-speed interface 1508 is coupled to the memory 1504, the display 1516 (e.g., through a graphics processor or accelerator), and to the high-speed expansion ports 1510, which may accept various expansion cards (not shown). In the implementation, the low-speed interface 1512 is coupled to the storage device 1506 and the low-speed expansion port 1514. The low-speed expansion port 1514, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1500 may be implemented in a number of different forms, as shown in the figure. For example, it may be implemented as a standard server 1520, or multiple times in a group of such servers. In addition, it may be implemented in a personal computer such as a laptop computer 1522. It may also be implemented as part of a rack server system 1524. Alternatively, components from the computing device 1500 may be combined with other components in a mobile device (not shown), such as a mobile computing device 1550. Each of such devices may contain one or more of the computing device 1500 and the mobile computing device 1550, and an entire system may be made up of multiple computing devices communicating with each other.

The mobile computing device 1550 includes a processor 1552, a memory 1564, an input/output device such as a display 1554, a communication interface 1566, and a transceiver 1568, among other components. The mobile computing device 1550 may also be provided with a storage device, such as a micro-drive or other device, to provide additional storage. Each of the processor 1552, the memory 1564, the display 1554, the communication interface 1566, and the transceiver 1568, are interconnected using various buses, and several of the components may be mounted on a common motherboard or in other manners as appropriate.

The processor 1552 can execute instructions within the mobile computing device 1550, including instructions stored in the memory 1564. The processor 1552 may be implemented as a chipset of chips that include separate and multiple analog and digital processors. The processor 1552 may provide, for example, for coordination of the other components of the mobile computing device 1550, such as control of user interfaces, applications run by the mobile computing device 1550, and wireless communication by the mobile computing device 1550.

The processor 1552 may communicate with a user through a control interface 1558 and a display interface 1556 coupled to the display 1554. The display 1554 may be, for example, a TFT (Thin-Film-Transistor Liquid Crystal Display) display or an OLED (Organic Light Emitting Diode) display, or other appropriate display technology. The display interface 1556 may comprise appropriate circuitry for driving the display 1554 to present graphical and other information to a user. The control interface 1558 may receive commands from a user and convert them for submission to the processor 1552. In addition, an external interface 1562 may provide communication with the processor 1552, so as to enable near area communication of the mobile computing device 1550 with other devices. The external interface 1562 may provide, for example, for wired communication in some implementations, or for wireless communication in other implementations, and multiple interfaces may also be used.

The memory 1564 stores information within the mobile computing device 1550. The memory 1564 can be implemented as one or more of a computer-readable medium or media, a volatile memory unit or units, or a non-volatile memory unit or units. An expansion memory 1574 may also be provided and connected to the mobile computing device 1550 through an expansion interface 1572, which may include, for example, a SIMM (Single In Line Memory Module) card interface. The expansion memory 1574 may provide extra storage space for the mobile computing device 1550, or may also store applications or other information for the mobile computing device 1550. Specifically, the expansion memory 1574 may include instructions to carry out or supplement the processes described above, and may include secure information also. Thus, for example, the expansion memory 1574 may be provided as a security module for the mobile computing device 1550, and may be programmed with instructions that permit secure use of the mobile computing device 1550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. The computer program product contains instructions that, when executed, perform one or more methods, such as those described above. The computer program product can be a computer- or machine-readable medium, such as the memory 1564, the expansion memory 1574, or memory on the processor 1552. In some implementations, the computer program product can be received in a propagated signal, for example, over the transceiver 1568 or the external interface 1562.

The mobile computing device 1550 may communicate wirelessly through the communication interface 1566, which may include digital signal processing circuitry where necessary. The communication interface 1566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1568 using a radio-frequency. In addition, short-range communication may occur, such as using a Bluetooth, WiFi, or other such transceiver (not shown). In addition, a GPS (Global Positioning System) receiver module 1570 may provide additional navigation- and location-related wireless data to the mobile computing device 1550, which may be used as appropriate by applications running on the mobile computing device 1550.

The mobile computing device 1550 may also communicate audibly using an audio codec 1560, which may receive spoken information from a user and convert it to usable digital information. The audio codec 1560 may likewise generate audible sound for a user, such as through a speaker, e.g., in a handset of the mobile computing device 1550. Such sound may include sound from voice telephone calls, may include recorded sound (e.g., voice messages, music files, etc.) and may also include sound generated by applications operating on the mobile computing device 1550.

The mobile computing device 1550 may be implemented in a number of different forms, as shown in the figure. For example, it may be implemented as a cellular telephone 1580. It may also be implemented as part of a smart-phone 1582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Although various implementations have been described in detail above, other modifications are possible. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

1. A method for providing personalized wellness recommendations, comprising:
obtaining epigenetic data for a person;
identifying wellness goals for the person;
determining one or more wellness recommendations for the person based on the epigenetic data and the wellness goals for the person; and
providing an indication of the one or more wellness recommendations.

2. The method of claim 1, further comprising:
obtaining genetic data for the person; and
determining the one or more wellness recommendations for the person further based on the genetic data.

3. The method of claim 2, wherein the genetic data comprises polygenic scores.

4. The method of claim 1, further comprising:
obtaining biographical information for the person; and
determining the one or more wellness recommendations for the person further based on the biographical information.

5. The method of claim 4, wherein the biographical information comprises information indicating at least one of an age, gender, ethnicity, height, weight, body-mass index (BMI), waist circumference, wrist circumference, blood glucose monitoring data, fitness tracking data, or medical history of the person.

6. The method of claim 1, wherein the epigenetic data describes a methylation pattern in DNA of the person.

7. The method of claim 6, wherein the DNA is extracted from a biological sample from the person, the biological sample comprising saliva, blood, or hair.

8. The method of claim 1, wherein the epigenetic data describes one or more epi-alleles in DNA of the person.

9. The method of claim 1, wherein the wellness goals are selected from a group comprising goals to live longer, have more energy, be happier, sleep better, increase muscle tone, improve cardiovascular fitness, reduce back, joint, or muscle pain, improve nutrition, lose weight, reduce or quit alcoholic drinking, and reduce or quit smoking.

10. The method of claim 1, further comprising:
assigning weights to the wellness goals; and
determining the wellness recommendations for the person using the weights for the wellness goals.

11. The method of claim 1, wherein determining the wellness recommendations for the person comprises determining recommendations for the person in one or more wellness categories.

12. The method of claim 11, further comprising selecting the one or more wellness categories from a plurality of wellness categories based on at least one of the wellness goals for the person, the epigenetic data for the person, biographical information for the person, or genetic data for the person.

13. The method of claim 11, wherein the wellness categories include at least one of a nutrition category, a fitness category, or a mindfulness category.

14. The method of claim 13, wherein the wellness categories include a nutrition category, and the wellness recommendations comprise a recommendation for a suggested nutrient, food, or food group for the person to consume to improve nutrition.

15. The method of claim 13, wherein the wellness categories include a fitness category, and the wellness recommendations comprise a recommendation for a suggested fitness activity or type of fitness activity for the person to engage in to improve fitness.

16. The method of claim 13, wherein the wellness categories include a mindfulness category, and the recommendations comprise a recommendation for a suggested mindfulness activity or mindfulness routine for the person to engage in to improve mindfulness.

17. The method of claim 1, further comprising determining a plurality of wellness recommendations for the person based on the epigenetic data and the wellness goals for the person, wherein providing the indication of the one or more wellness recommendations comprises ranking the plurality of wellness recommendations for the person based on the epigenetic data and the wellness goals for the person.

18. The method of claim 17, further comprising:
ordering the plurality of wellness recommendations according to the ranking of the plurality of wellness recommendations; and
providing a report of the wellness recommendations for the person according to the order of the plurality of wellness recommendations.

19-30. (canceled)

31. One or more non-transitory computer-readable media having instructions stored thereon that, when executed by one or more processors, cause the one or more processors to perform operations comprising:
obtaining epigenetic data for a person;
identifying wellness goals for the person;
determining one or more wellness recommendations for the person based on the epigenetic data and the wellness goals for the person; and
providing an indication of the one or more wellness recommendations.

32. A system, comprising:
one or more processors; and
one or more computer-readable media having instructions stored thereon that, when executed by the one or more processors, cause the one or more processors to perform operations comprising:
obtaining epigenetic data for a person;
identifying wellness goals for the person;
determining one or more wellness recommendations for the person based on the epigenetic data and the wellness goals for the person; and
providing an indication of the one or more wellness recommendations.
